(19) United States
(12) Patent Application Publication
(10) Pub. No.: US 2025/0002435 A1
(43) Pub. Date: Jan. 2, 2025

(54) ARYLATED FENCHONE DERIVATIVES, COMPOSITIONS COMPRISING THEM, PROCESSES FOR THEIR PREPARATION AND USES THEREOF

(71) Applicant: Yissum Research Development Company of The Hebrew University of Jerusalem Ltd., Jerusalem (IL)

(72) Inventors: Raphael MECHOULAM (deceased), Jerusalem (IL); Reem SMOUM, Beit Hanina (IL); Ruth GALLILY, Jerusalem (IL); Mona DVIR-GINZBERG, Modiin (IL)

(73) Assignee: Yissum Research Development Company of The Hebrew University of Jerusalem Ltd., Jerusalem (IL)

(21) Appl. No.: 18/699,294

(22) PCT Filed: Oct. 7, 2022
(86) PCT No.: PCT/IL22/51073

§ 371 Date: Apr. 7, 2024

Related U.S. Application Data
(60) Provisional application No. 63/254,130, filed on Oct. 10, 2021.
(60) Provisional application No. 63/296,201, filed on Jan. 4, 2022.

(51) Int. Cl. C07C 43/23; A61K 31/085; A61K 31/09; A61P 19/02

(52) U.S. Cl. CPC C07C 43/23; A61K 31/085; A61K 31/09; A61P 19/02; C07C 2602/42

(57) ABSTRACT

The present invention provides arylated fenchone derivatives, compositions comprising them, processes for their preparation and uses thereof.

### BACKGROUND OF THE INVENTION

Terpenoids are the largest group of natural compounds. They have biological activities including antimicrobial, antitumor and anti-inflammatory and are used for the treatment of human diseases such as cancer, malaria, inflammation, and a variety of infectious diseases (viral and bacterial). Fenchone, a natural organic compound classified as a monoterpene and a ketone is a constituent of absinthe and the essential oil of fennel. It was one of the detected and fully identified components of the volatile oil of fresh and air-dried buds of Cannabis sativa. Fenchone exerts anti-inflammatory action in rats by reducing inflammation in the carrageenan-induced right hind-paw edema model. Also, being a major constituent of Foeniculum vulgare essential oil, fenchone proved antinociceptive activity in tail-flick model of pain in mice without inducing motor incoordination. Recent data have demonstrated the protective effects of the Lavandula stoechas essential oils, where the principal compound is D-fenchone (29.28%), against diabetes and oxidative stress induced by alloxan treatment in rat. Lavender essential oils decreased kidney and hepatic injuries through their antioxidant properties and played a major role as hepato- and nephroprotection products.

The monoterpene groups as well as the 2,5-disubstituted resorcinols are essential components in the synthesis of cannabinoids. The endocannabinoid system (ECS) is an emerging target for the regulation of inflammation and the immune response. The ECS activation occurs via ligand binding to the cannabinoid receptors 1 (CB1R) and 2 (CB2R). CB1R is mainly expressed in the central nervous system (CNS) and mediates the classical psychotropic effects, the CB2R is primarily expressed in the periphery where it is found on cells of the immune system and exerts anti-inflammatory effects. CB2R has become an attractive target since it lacks the adverse psychotropic effects associated with CB1R activation and has limited expression in the peripheral tissues. It has a key regulatory role in inflammation and modulating immune responses. CB2 receptor activation inhibits upstream and downstream molecules of the inflammatory process and its stimulation exerts analgesic activity. It is up-regulated in pathological conditions correlated with the onset of inflammatory events like cancer and neurodegenerative diseases.

### SUMMARY OF THE INVENTION

The present invention provides a compound having the general formula (I), including any stereoisomer, salt or solvate thereof:

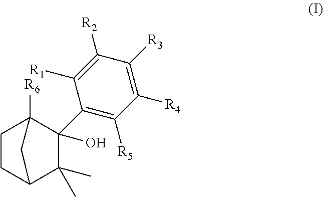

Wherein R1 and R5 are each selected from H, OH, and —O(straight or branched C1-C5 alkyl); provided that at least one of R1 and R5 is different than H; and wherein R2, R3 and R4 are each selected from H, straight or branched C1-C10 alkyl, straight or branched C2-C10 alkenyl, straight or branched C2-C10 alkynyl, halogen (F, Cl, Br, I); provided that at least one of R2, R3 and R4 are different than H; wherein R6 is selected from straight or branched C1-C5 alkyl, —CH2OH, —COOH, —COO(straight or branched C1-C5 alkyl), —C(═O)H, —C(═O)(straight or branched C1-C5 alkyl).

In some embodiments, R1 and R5 are each O(straight or branched C1-C5 alkyl).

In some embodiments, at least one of R1 and R5 is O(straight or branched C1-C5 alkyl).

In some embodiments, at least one of R1 and R5 is OH.

In some embodiments, at least one of R2, R3 and R4 is halogen. In some embodiments, said halogen is F. In some embodiments, said halogen is Cl. In some embodiments, said halogen is Br. In some embodiments, said halogen is I.

In some embodiments, at least one of R2, R3 and R4 is a straight or branched C3-C10 alkyl.

In some embodiments, R1 and R5 are each O(straight or branched C1-C5 alkyl) and R3 is straight or branched C3-C10 alkyl.

In some embodiments, R1 and R5 are each O(straight or branched C1-C5 alkyl) and R4 is straight or branched C3-C10 alkyl.

In some embodiments, R1 and R5 are each O(straight or branched C1-C5 alkyl) and R2 is a halogen.

In some embodiments, R1 and R5 are each O(straight or branched C1-C5 alkyl) and R2 is a halogen and R3 is a straight or branched C3-C10 alkyl.

In some embodiments, R1 is O(straight or branched C1-C5 alkyl) and R5 is H.

In some embodiments, R1 is O(straight or branched C1-C5 alkyl) and R5 is H and R4 is a straight or branched C3-C10 alkyl.

In some embodiments, R1 is O(straight or branched C1-C5 alkyl) and R5 is H and R4 is a straight or branched C3-C8 alkyl.

In some embodiments, R1 is O(straight or branched C1-C5 alkyl) and R5 is OH and R3 is a straight or branched C3-C8 alkyl.

In some embodiments R1 is selected from H, OH, and —O(straight or branched C1-C5 alkyl). In some embodiments R1 is H. In some embodiments R1 is OH. In some embodiments R1 is —O(straight or branched C1-C5 alkyl).

In some embodiments R5 is selected from H, OH, and —O(straight or branched C1-C5 alkyl). In some embodiments R5 is H. In some embodiments R5 is OH. In some embodiments R5 is —O(straight or branched C1-C5 alkyl).

In some embodiments R2 is selected from H, straight or branched C1-C10 alkyl, straight or branched C2-C10 alkenyl, straight or branched C2-C10 alkynyl, halogen (F, Cl, Br, I). In some embodiments R2 is H. In some embodiments R2 is straight or branched C1-C10 alkyl. In some embodiments R2 is straight or branched C2-C10 alkenyl. In some embodiments R2 is straight or branched C2-C10 alkynyl. In some embodiments R2 is halogen (selected from F, Cl, Br, I).

In some embodiments R3 is selected from H, straight or branched C1-C10 alkyl, straight or branched C2-C10 alkenyl, straight or branched C2-C10 alkynyl, halogen (F, Cl, Br, I). In some embodiments R3 is H. In some embodiments R3 is straight or branched C1-C10 alkyl. In some embodiments R3 is straight or branched C2-C10 alkenyl. In some embodiments R3 is straight or branched C2-C10 alkynyl. In some embodiments R2 is halogen (selected from F, Cl, Br, I).

In some embodiments R4 is selected from H, straight or branched C1-C10 alkyl, straight or branched C2-C10 alkenyl, straight or branched C2-C10 alkynyl, halogen (F, Cl, Br, I). In some embodiments R4 is H. In some embodiments R4 is straight or branched C1-C10 alkyl. In some embodiments R4 is straight or branched C2-C10 alkenyl. In some embodiments R4 is straight or branched C2-C10 alkynyl. In some embodiments R4 is halogen (selected from F, Cl, Br, I).

In some embodiments R6 is selected from straight or branched C1-C5 alkyl, —CH2OH, —COOH, —COO(straight or branched C1-C5 alkyl), —C(═O)H, —C(═O)(straight or branched C1-C5 alkyl).

In some embodiments R6 is a straight or branched C1-C5 alkyl. In other embodiments, R6 is CH3. In further embodiments, R6 is selected from —CH2OH, —COOH, —COO(straight or branched C1-C5 alkyl), —C(═O)H, —C(═O)(straight or branched C1-C5 alkyl). In some embodiments, R6 is —CH2OH. In some embodiments, R6 is —COOH. In some embodiments, R6 is —COO(straight or branched C1-C5 alkyl). In some embodiments, R6 is —C(═O)H. In some embodiments, R6 is —C(═O)(straight or branched C1-C5 alkyl).

When referring to C1-C10 alkyl or C1-C5 alky it should be understood to encompass a straight or branched hydrocarbon chain that has 1, 2, 3, 4, 5, 6, 7, 8, 9, or 10 carbon atoms all connected to one another with a single sigma bond.

When referring to straight or branched C2-C10 alkenyl it should be understood to encompass a straight or branched hydrocarbon chain that has 2, 3, 4, 5, 6, 7, 8, 9, or 10 carbon atoms comprising at least one double π bond and all other bonds being single sigma bonds.

When referring to straight or branched C2-C10 alkynyl it should be understood to encompass a straight or branched hydrocarbon chain that has 2, 3, 4, 5, 6, 7, 8, 9, or 10 carbon atoms comprising at least one triple bond and all other bonds being single sigma bonds.

In some embodiments, a compound of the invention is selected from:

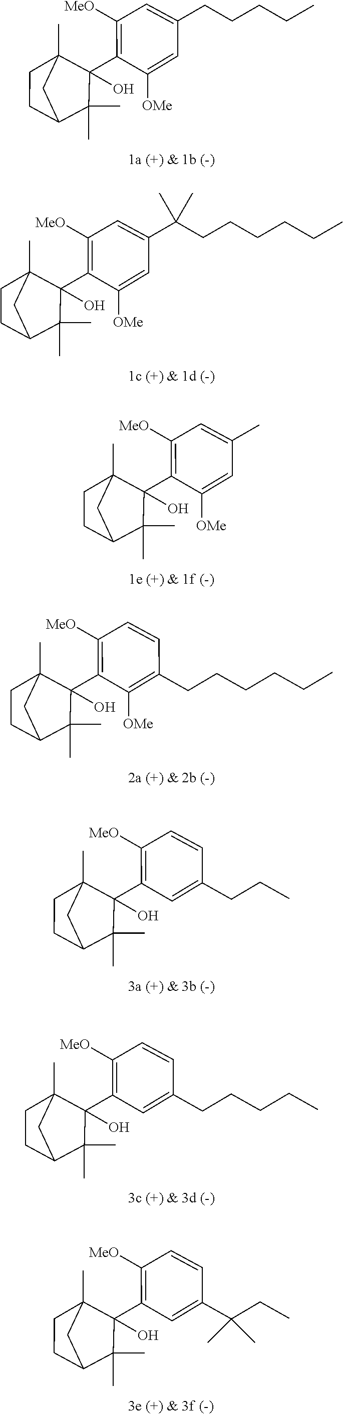

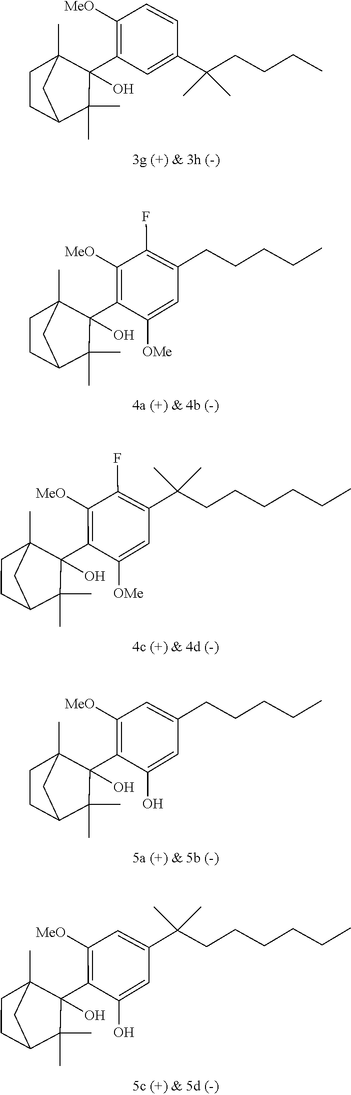 [SMILES: CCCCC(C)(C)c1ccc(OC)c(C2(O)C3(C)CCC(C3)C2(C)C)c1 ; CCCCCc1cc(OC)c(C2(O)C3(C)CCC(C3)C2(C)C)c(OC)c1F ; CCCCCCC(C)(C)c1cc(OC)c(C2(O)C3(C)CCC(C3)C2(C)C)c(OC)c1F ; CCCCCc1cc(O)c(C2(O)C3(C)CCC(C3)C2(C)C)c(OC)c1 ; CCCCCCC(C)(C)c1cc(O)c(C2(O)C3(C)CCC(C3)C2(C)C)c(OC)c1]

It is to be understood that when referring to a “stereoisomer” it encompasses any stereochemical isomer of a compounds provided herein that contain one or more chiral centers (enantiomers or diastereomers accordingly). Such chiral centers may be of either the (R) or (S), (D) or (L) configuration or may be a mixture thereof. Thus, the compounds provided herein may be enantiomerically pure, or be stereoisomeric or diastereomeric mixtures. Furthermore, this term includes designating the compound of the invention by the optical activity of a pure or a mixture of compounds being (+), (−) or (±). In case a compound of the invention further includes an asymmetric double bond, the term also includes any of the cis-, trans-, E- or Z-designations of the compound.

As used herein, the term “solvate” refers to a complex of variable stoichiometry formed by a solute (in this invention, a compound of the invention or a salt or physiologically functional derivative thereof) and a solvent. Such solvents for the purpose of the invention may not interfere with the biological activity of the solute.

The invention also includes any salt of a compound of formula (I), including any pharmaceutically acceptable salt, wherein a compound of the invention has a net charge (either positive or negative) and at least one counter ion (having a counter negative or positive charge) is added thereto to form said salt. The phrase “pharmaceutically acceptable salt(s)”, as used herein, means those salts of compounds of the invention that are safe and effective for pharmaceutical use in mammals and that possess the desired biological activity. Pharmaceutically acceptable salts include salts of acidic or basic groups present in compounds of the invention.

The invention further provides a pharmaceutical composition comprising at least one compound as disclosed herein above and below. Said at least one compound as disclosed herein above and below being the active pharmaceutical agent (as active pharmaceutical ingredient (API) defined as the substance in a pharmaceutical composition that is/are responsible for the biological effects in the methods of treatment disclosed herein).

The present invention also relates to pharmaceutical compositions comprising at least one compound of the subject invention in admixture with pharmaceutically acceptable auxiliaries, and optionally other therapeutic agents. The auxiliaries must be “acceptable” in the sense of being compatible with the other ingredients of the composition and not deleterious to the recipients thereof.

Pharmaceutical compositions include those suitable for oral, rectal, nasal, topical (including transdermal, buccal and sublingual), vaginal or parenteral (including subcutaneous, intramuscular, intravenous and intradermal) administration or administration via an implant. The compositions may be prepared by any method well known in the art of pharmacy.

Such methods include the step of bringing in association compounds used in the invention or combinations thereof with any auxiliary agent. The auxiliary agent(s), also named accessory ingredient(s), include those conventional in the art, such as carriers, fillers, binders, diluents, disintegrants, lubricants, colorants, flavouring agents, anti-oxidants, and wetting agents.

Pharmaceutical compositions suitable for oral administration may be presented as discrete dosage units such as pills, tablets, dragées or capsules, or as a powder or granules, or as a solution or suspension. The active ingredient may also be presented as a bolus or paste. The compositions can further be processed into a suppository or enema for rectal administration.

The invention further includes a pharmaceutical composition, as herein before described, in combination with packaging material, including instructions for the use of the composition for a use as herein before described.

For parenteral administration, suitable compositions include aqueous and non-aqueous sterile injection. The compositions may be presented in unit-dose or multi-dose containers, for example sealed vials and ampoules, and may be stored in a freeze-dried (lyophilised) condition requiring only the addition of sterile liquid carrier, for example water, prior to use. For transdermal administration, e.g. gels, patches or sprays can be contemplated. Compositions or formulations suitable for pulmonary administration e.g. by nasal inhalation include fine dusts or mists which may be generated by means of metered dose pressurized aerosols, nebulisers or insufflators.

The exact dose and regimen of administration of the composition will necessarily be dependent upon the therapeutic or nutritional effect to be achieved and may vary with the particular formula, the route of administration, and the age and condition of the individual subject to whom the composition is to be administered.

The invention further provides a compound as disclosed herein above and below, being a CB2 receptor agonist.

The invention further provides a compound as disclosed herein above and below, for use in the treatment of a disease, condition or disorder associated with CB2 receptor.

The invention further provides a compound as disclosed herein above and below, for use in the treatment of at least one disease, condition or disorder selected from inflammation, pain, autoimmune disease, neurological and neurodegenerative disease, liver disease, multiple sclerosis, osteoporosis, osteoarthritis, CNS disorder, cancer and any combinations thereof.

The invention further provides a compound as disclosed herein above and below, for use in the treatment of inflammation. The invention further provides a compound as disclosed herein above and below, for use in the treatment of pain. The invention further provides a compound as disclosed herein above and below, for use in the treatment of an autoimmune disease. The invention further provides a compound as disclosed herein above and below, for use in the treatment of neurological and neurodegenerative disease. The invention further provides a compound as disclosed herein above and below, for use in the treatment of liver disease. The invention further provides a compound as disclosed herein above and below, for use in the treatment of multiple sclerosis. The invention further provides a compound as disclosed herein above and below, for use in the treatment of osteoporosis. The invention further provides a compound as disclosed herein above and below, for use in the treatment of osteoarthritis. The invention further provides a compound as disclosed herein above and below, for use in the treatment of CNS disease and/or disorder. The invention further provides a compound as disclosed herein above and below, for use in the treatment of cancer.

The invention further provides a method of treating a subject suffering from a disease, condition or disorder associated with CB2 receptor; said method comprising administering to said subject at least one compound as disclosed herein above and below.

The term “treatment” as used herein means the management and care of a patient for the purpose of combating a disease, disorder or condition. The term is intended to include the delaying of the progression of the disease, disorder or condition, the alleviation or relief of symptoms and complications, and/or the cure or elimination of the disease, disorder or condition. The patient to be treated is preferably a mammal, in particular a human being.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows the synthetic routes of compounds 1a-1f.

FIG. 2 shows the synthetic routes of compounds 2a-2b.

FIG. 3 shows the synthetic routes of compounds 3a-3h.

FIG. 4 shows the synthetic routes of compounds 4a-4d.

FIG. 5 show the synthetic routes of compounds 5a-5d.

FIG. 6 show the HSQC of compound 1d.

FIGS. 7A and 7B show the crystal structures of 1b (7A) and 4b (7B).

FIGS. 8A and 8B show the crystal structures of 1d (8A) and 5d (8B). FIG. 8C shows the orientation of docked ligands in the orthosteric site of CB2. Binding site of the CB2 cavity is represented by electrostatic potential surface. Ligands are shown in different colors; 5ZTY_ligand (pink), HU-308 (Yellow), HU-433 (Orange) and 1d (Green). TM means Transmembrane Helix. The residues of binding site and ligands are represented by thin and thick tubes respectively. H-bonds and pi-pi interactions are represented by orange and cyan dotted lines respectively.

FIGS. 9A, 9B and 9C relates to displacement of [³H]CP-55940 by HU-308, 1b and 1d from specific binding sites in membranes from cells expressing hCB2Rs. Each symbol represents the mean percent displacement ±SE (9A); Structural Requirements for CB2 affinity and selectivity (9B); Mean log concentration-response curves of 1b and 1d for stimulation of [³S]GTPγS binding to hCB2R CHO cell membranes. Each symbol represents the mean percentage increase in [³⁵S]GTPγS binding ±SE (9C).

FIGS. 10A-10D show the anti-inflammatory and anti-nociceptive effects of 5b or 5d. (10A) (10B) Prevention of zymosan-induced swelling of hind paw. 1.5% zymosan in 40 μl was injected into the sub-planter surface of the right hind paw. Immediately thereafter, 5b (10A) or 5d (10B) was injected intraperitoneally. The paw thickness indicative for paw swelling was measured 2, 6 and 24 h thereafter. The paw thickness of untreated mice was 2.0-2.3 mm, which made the baseline of the graph. N=12 for each time point. *p<0.05 compared to control mice; (10C) (10D) Antipain effect of 5b (10C) and 5d (10D). The hyperalgesia was measured by using the von Frey nociceptive filament assay. The higher the paw withdrawal threshold, the higher is the anti-nociceptive effect of the drug. The experiments were repeated three times, each experiment with 4 mice in each treatment group. The graphs present the average of all mice in the three experiments, meaning that the N=12 for each time point. The bars represent standard error. *p<0.05 compared to control mice.

FIGS. 11: Fenchone 1D and 1B are selective agonists for CNR2. (11A) To test for agonist activity of the GPCR cells expressing CNR1 or CNR2 were exposed to Forskolin (positive control), 1B and 1D Fenchones and assayed for EC50 values, as in the Materials and Methods (Table SD1). (11B) Antagonistic activity was assessed using forskulin (Agonist control CNR1, CNR2) and antagonist controls (AM281 for CNR1 and SR144528 for CNR2), as well as 1D and 1B Fenchone compounds (Table SD2). (11C) Human OA derived articular chondrocytes were plated at confluence and treated with HU308, 1D, 1B Fenchones for 45 min and assessed for EC50 values. The values were obtained using GraphPad Prism EC50 curve fits which display the minimum and maximum ranges along with the Hilslope fit value.

FIGS. 12A-12D shows the assessment of 1D/1B effects in a mouse paw inflammatory pain model. Mice were induced with zymosan and treated as detailed in materials and methods (12A). (12B) exhibits swelling and pain (left and right graphs respectively) of mice treated with 25 mg/kg (IP) 1B, 1D and/or a CB2 antagonist 1 mg/kg SR144528. (12C) exhibits swelling and pain (left and right graphs respectively) of mice treated with 25 mg/kg (IP) 1D, and/or 10 mg/kg AM630. Statistical significance between treatments and vehicle control (denoted “*”) OR between treatments and 1D control (denoted “#”), were examined via Kruskal-Walis test with a Dunn's post-hoc analysis for scored (non-parametric) parameters, considering p<0.05 to be statistically significant (n=3).

FIGS. 13A-13D shows the MMT experimental setup and behavioral phenotyping: (13A) Experimental setup of MMT procedure carried out in the right hindlimb of Lewis rats. Five groups were examined (Sham, n=5; MMT-Vehicle n=15; MMT-1D, low dose 8 ug/mL n=15; MMT-1D, high dose 24 ug/mL n=15 and MMT-FGF18 60 ug/mL n=15). Vehicle and 1D groups were administered intra-articularly (IA) at 4,7,10, 14, 17, 21, 24 days post-MMT, while FGF18 group was administered IA at 7, 14, 21 post-procedure. All mice were weighed (13B) and subjected to dynamic weigh bearing differences (Left to right hindlimbs) (13C) at 4, 7, 14 and 28 days post MMT, prior to their sacrifice. (13D) Area under the curve of dynamic weigh bearing (DWB) between 4 and 28 days post MMT. Statistical significance between treatments and control, were examined via Kruskal-Walis test with a Dunn's post-hoc analysis for scored (non-parametric) parameters, considering p<0.05 (*) to be statistically significant. **p<0.01; ***p<0.001; ****p<0.0001.

FIGS. 14A-14C: MMT Histopathological Profiling and Cartilage Degenerative scores: (14A) Exhibits the post sacrifice histopathological scores employed for sagittal section of the medial tibial plateau, including (a) cartilage zonal and total degenerative scores; (b) Zonal and average depth ratios; (c) Synovitis scores; (d) Osteophyte measurements and scores and (e) calcified cartilage damage scores. (14B) Represents cartilage zonal and total degenerative scores for all five experimental groups. These scores exhibit the zone related and cumulative zone related damage as per scoring table in SD3 criteria, and based on toluidine blue stained sections (14C, representative sections shown). Statistical significance between treatments and control, were examined via Kruskal-Walis test with a Dunn's post-hoc analysis for scored (non-parametric) parameters, considering p<0.05 (*) to be statistically significant. **p<0.01; ***p<0.001; ****p<0.0001. Sham, n=5; MMT-Vehicle n=15; MMT-1D, low dose 8 ug/mL n=15; MMT-1D, high dose 24 ug/mL n=15 and MMT-FGF18 60 ug/mL n=15.

FIGS. 15A-15B shows the histopathology for post-MMT Medial Tibial Degeneration Depth ratio. The zonal depth ratio of toluidine stained sections is shown in (15A) for all 5 groups, per zone of the medial tibial plateau and per a mean of the three zones. (15B) exhibits the method for obtaining depth ratios, for the measured depth of mid area of the zone vs the total anticipated area should damage have no occurred. High rations indicate more extensive damage than low ratios. Statistical significance between treatments and control, were examined via Kruskal-Walis test with a Dunn's post-hoc analysis for scored (non-parametric) parameters, considering p<0.05 (*) to be statistically significant. **p<0.01; ***p<0.001; ****p<0.0001. Sham, n=5; MMT-Vehicle n=15; MMT-1D, low dose 8 ug/mL n=15; MMT-1D, high dose 24 ug/mL n=15 and MMT-FGF18 60 ug/mL n=15.

FIGS. 16A-16B shows histopathology for post-MMT Medial Tibial Synovitis and Osteophyte profiles. Synovitis scores were assessed as per SD4, and displayed for all five groups (16A) with a repressive scheme and repressive images (right panels). (16B) Exhibits the measurement of osteophytes (left graph and upper illustration) and the score as per SD5 (right graph). The representative images are show to the left of the graphs in panel B. Statistical significance between treatments and control, were examined via Kruskal-Walis test with a Dunn's post-hoc analysis for scored (non-parametric) parameters, considering p<0.05 (*) to be statistically significant. **p<0.01; ***p<0.001; ****p<0.0001. Sham, n=5; MMT-Vehicle n=15; MMT-1D, low dose 8 ug/mL n=15; MMT-1D, high dose 24 ug/mL n=15 and MMT-FGF18 60 ug/mL n=15.

FIGS. 17A-17C show histopathology for post-MMT Medial Tibial Bone damage. Bone damage is illustrated in (A) and exhibited in graphs B. (C) exhibits representative sections showing the relevant bone morphology for each group Statistical significance between treatments and control, were examined via Kruskal-Walis test with a Dunn's post-hoc analysis for scored (non-parametric) parameters, considering p<0.05 (*) to be statistically significant. **p<0.01; ***p<0.001; ****p<0.0001. Sham, n=5; MMT-Vehicle n=15; MMT-1D, low dose 8 ug/mL n=15; MMT-1D, high dose 24 ug/mL n=15 and MMT-FGF18 60 ug/mL n=15.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Fenchone is a bicyclic monoterpene present in essential oils of plant species and is a component of the volatile oil of fresh and air-dried buds of Cannabis sativa. It exerts anti-inflammatory action in rats as noted in a carrageenan-induced right hind-paw edema model. Also, being a major constituent of Foeniculum vulgare essential oil, fenchone was shown to have an anti-nociceptive activity in the tail-flick pain mouse model, without inducing motor incoordination. The protective effects of Lavandula stoechas essential oil, where the principal compound is D-fenchone (29.28%), against diabetes and oxidative stress induced by alloxan treatment in rats. Lavender essential oils also decrease kidney and hepatic injuries through their antioxidant properties and play a major role as hepato- and nephroprotection products.

Monoterpenes and 5-substituted resorcinols are widely used for syntheses of cannabinoids. Many of them modulate the endocannabinoid system (ECS), which is an emerging target for the regulation of inflammation and the immune response. ECS activation occurs either via ligands binding to the cannabinoid receptors 1 (CB1R) and 2 (CB2R) or in an indirect way, by promoting the synthesis of endocannabinoids, or, alternatively, in inhibiting their degradation. CB1R is mainly expressed in the central nervous system (CNS) and mediates the classical psychotropic effects, whereas the CB2R is primarily expressed in the periphery, where it is found on cells of the immune system, such as monocytes/macrophages, B cells, certain T-cell subtypes, and mast cells. CB2R has become an attractive target since it does not cause the adverse psychotropic effects associated with CB1R activation. Activation of the CB2R inhibits upstream and downstream molecules of the inflammatory process, and its stimulation exerts analgesic activity. It is up regulated in pathological conditions correlated with the onset of inflammatory events in cancer and neurodegenerative diseases. CB2 agonists restrain inflammatory responses in hepatic ischemia-reperfusion injury, uveitis, and contact dermatitis. Some synthetic agonists, such as HU-308, JWH-133, and HU-910 have terpene and resorcinol-derived moieties in their structure and hence they resemble the phytocannabinoids A9-tetrahydrocannabinol (A9-THC) and cannabidiol (CBD). Other, non-phytocannabinoid-type agonists have also been reported.

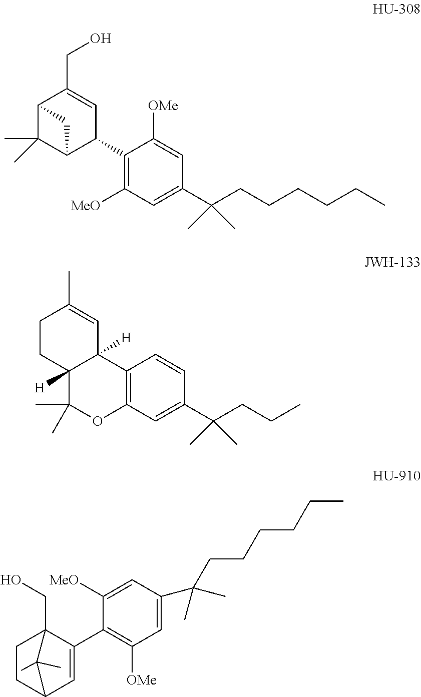

The synthesis and structural identification of twenty-four novel bicyclic monoterpenoid fenchone derivatives with different alkylresorcinol and alkylphenyl groups are presented herein. This was started off by the synthesis of fenchone-alkylresorcinols and fenchone-alkylphenols (Substitution I) in alignment with the previously reported HU-308, JWH-133 and HU-910. Next, the effect of fluorination of the aromatic ring in the fenchone-alkylresorcinols (Substitution II) bearing different aliphatic substituents was explored. Then, the fenchone-alkylresorcinols with different alkyl substituents were demethylated (Substitution III). The structures of the compounds were characterized by NMR, GCMS and LC-UV-MS (ESI). 1D and 2D NMR experiments (DEPT, gCOSY, TOCSY, HSQC and HMBC) were used to determine the structure assignment of three different fenchone derivatives. Single-crystal X-ray diffraction was used to determine the absolute configuration of four derivatives. All synthesized compounds were assessed for their binding affinities at human CB1R (hCB1R) and human CB2R (hCB2R). Affinity data (Ki values) were used to calculate the selectivity indices of these compounds. These ligands were also examined in the [35S]GTPγS binding assay with the aim of evaluation of their functional activity. To assess the in vivo efficacy of the newly developed chemotypes, two compounds from the most potent series were selected to be tested for their anti-inflammatory and anti-nociceptive properties. In addition, molecular-modeling studies were carried out to understand the binding interactions of 1d within the CB2 binding site and compare with the parent CB2 compounds.

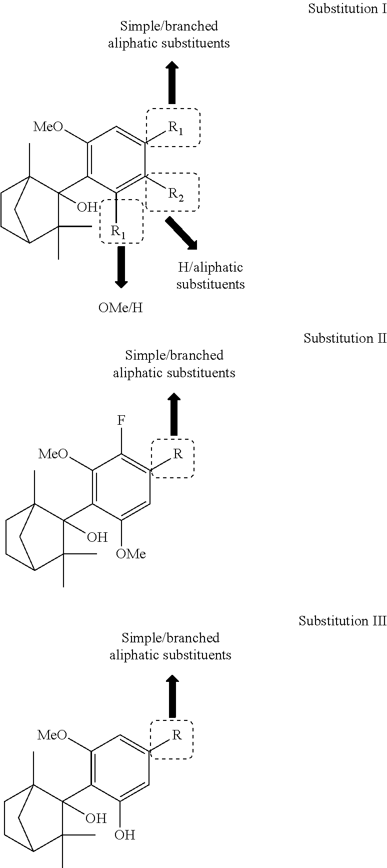

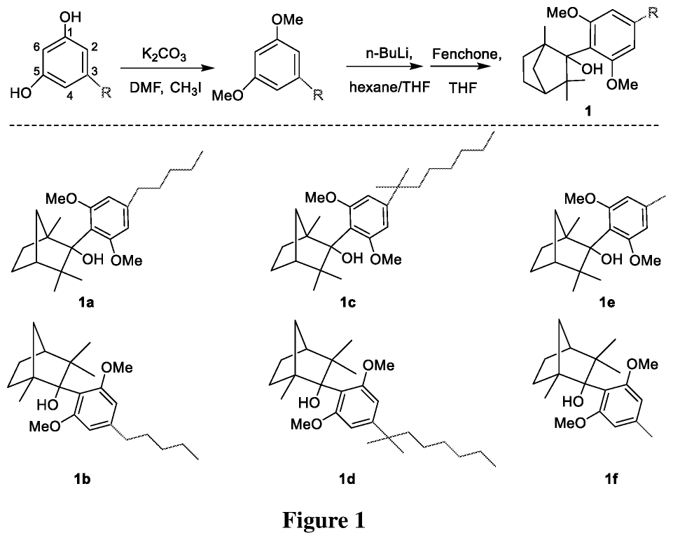

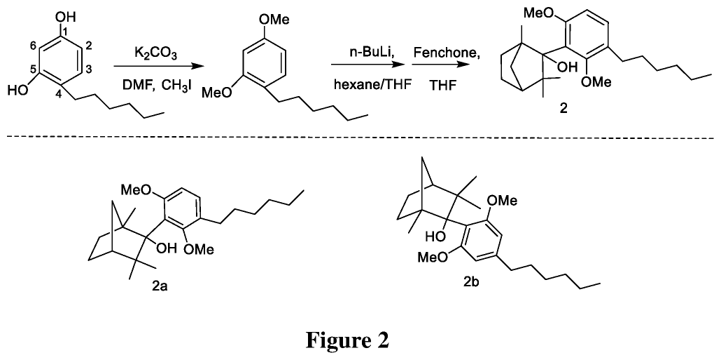

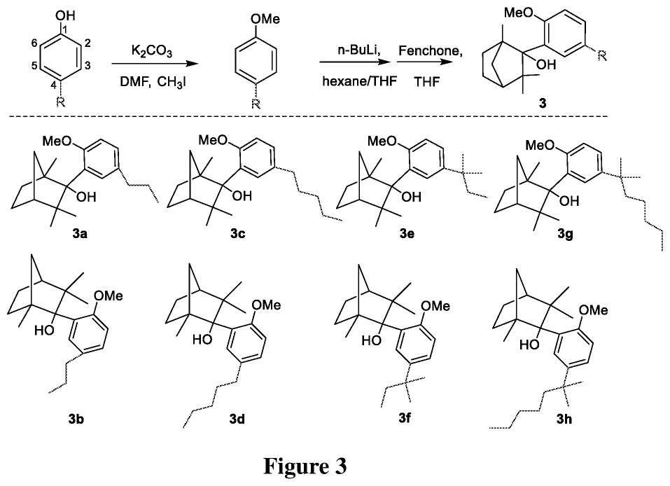

Chemistry: The fenchone-resorcinol/phenol derivatives were prepared using a three-step sequence. The first step was the methylation of 3-alkyl resorcinols/4-alkyl resorcinol/4-alkylphenols (FIGS. 1-3) using potassium carbonate, dimethylformamide and methyl iodide. Then the 6-lithio derivative of the corresponding resorcinol dimethyl ether/phenol methyl ether were prepared using n-butyllithium/hexane in THF. The final step was the condensation with the fenchone to give the final products (FIG. 1 for 1,5-dimethoxy-3-alkyl resorcinols, FIG. 2 for 1,5-dimethoxy-4-hexyl resorcinol and FIG. 3 for 4-alkylphenols). Different resorcinols/phenols required different reaction conditions for lithiation and condensation. For example, some compounds required lithiation under reflux for 2.5 h, followed by condensation for 3 h under reflux and then for 18 h at r.t. while other compounds required lithiation at 0° C. for 1 h and then condensation for 0.5 h at 0° C. and then for 18 h at r.t.

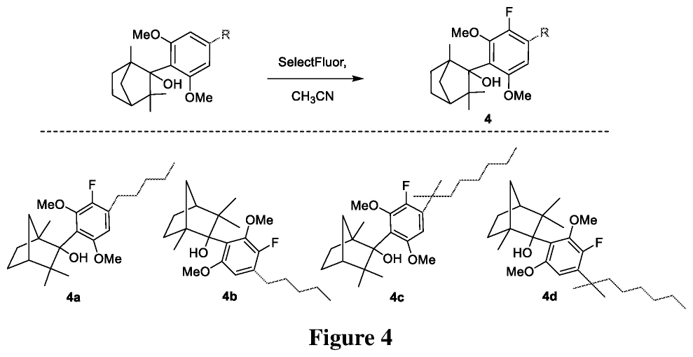

Introducing fluorine into such molecules can productively influence conformation, pKa, intrinsic potency, membrane permeability, metabolic pathways, and pharmacokinetic properties. Therefore, fluorinated compounds at the aromatic ring of the fenchone derivatives (1a-d) were synthesized (FIG. 4). They were obtained by the reaction of the fenchone-resorcinol with 1-Chloromethyl-4-fluoro-1,4-diazoniabicyclo[2.2.2]octane bis(tetra-fluoroborate) or Selectfluor. The optimal conditions involved the use of the Selectfluor reagent in MeCN at room temperature under nitrogen atmosphere. Selectfluor is one of the most reactive electrophilic fluorination reagents and is safe, nontoxic, and easy to handle. However, Selectfluor only works with resorcinol dimethylether derivatives. Fluorination of the fenchone derivatives with monomethoxy alkylphenyl substituents was not successful.

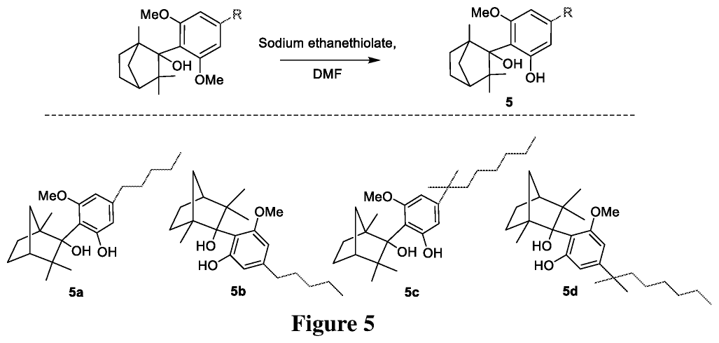

Demethylation of aromatic compounds involves the use of acids; however, fenchone upon treatment with acids undergoes rearrangement. Therefore, the dimethoxy alkylphenyl fenchol derivatives were demethylated with sodium thioethoxide in N,N-dimethylformamide. However, only one methoxyl group was demethylated (FIG. 5). Moreover, when trying to demethylate the methoxyalkylphenyl fenchol derivatives, no reaction occurred, meaning that this reagent works only with dimethoxy derivatives. In total, 24 novel fenchone-based compounds were synthesized and have been grouped in such a manner as to illustrate the effects of systematic structural variation.

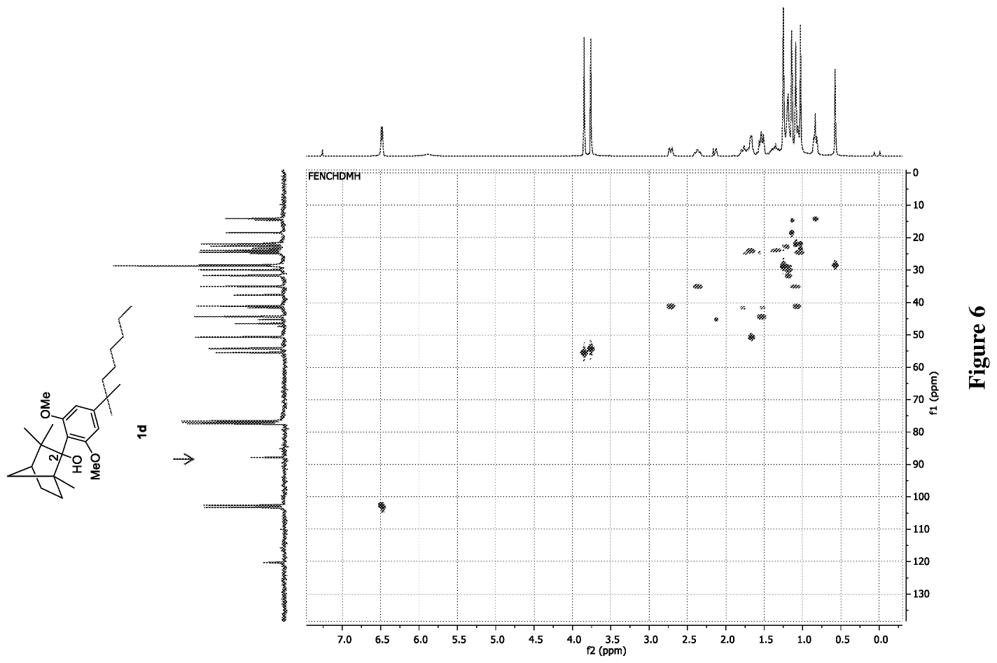

NMR Analysis: The structures of all compounds were determined by ¹H and ¹³C NMR and for the fluorinated compounds, ¹⁹F NMR was done. However, a complete analysis of 1D and 2D NMR spectra was performed for compound 1d. Its structure was assigned based on the analysis of ¹H, ¹³C, DEPT, gCOSY, TOCSY and gHSQC NMR. Through NMR analysis, it was possible to determine all the chemical shifts for all the carbons and hydrogens. The 2D HSQC permits to obtain a 2D heteronuclear chemical shift correlation map between directly-bonded 1H and X-heteronuclei (commonly, ¹³C and 15N). Here ¹H-¹³C-HSQC experiment (FIG. 6) was done and it was shown that Carbon 2 (of the fenchone) that is connected to a hydroxyl group does not have any cross peaks with the hydrogens and is shifted downfield.

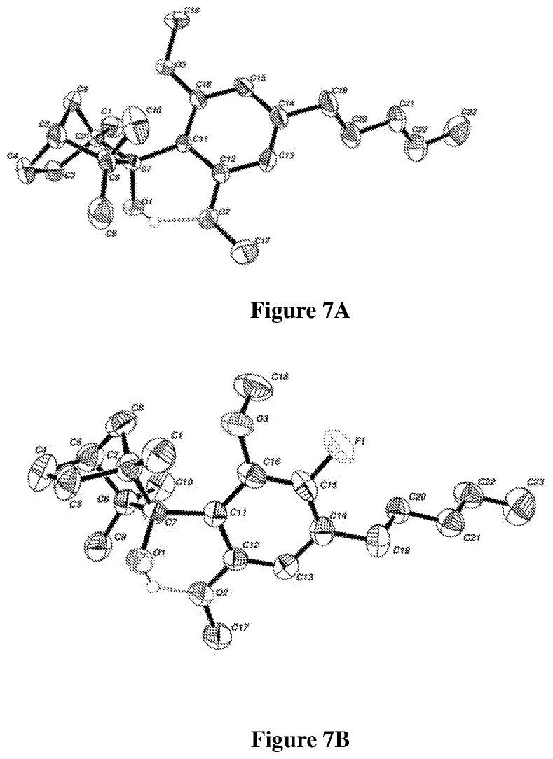

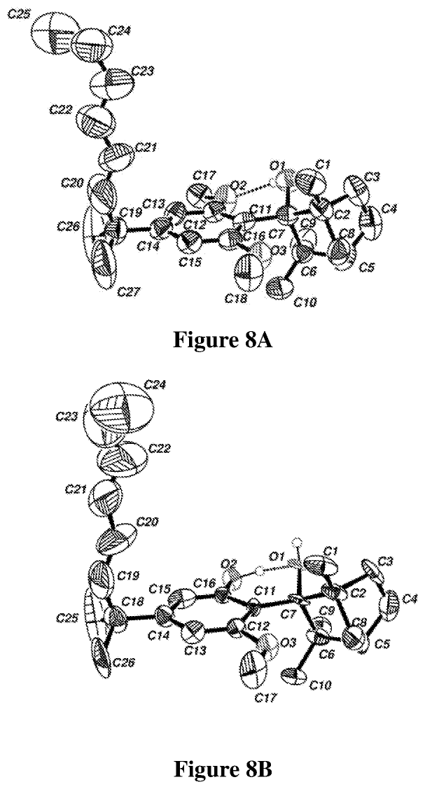

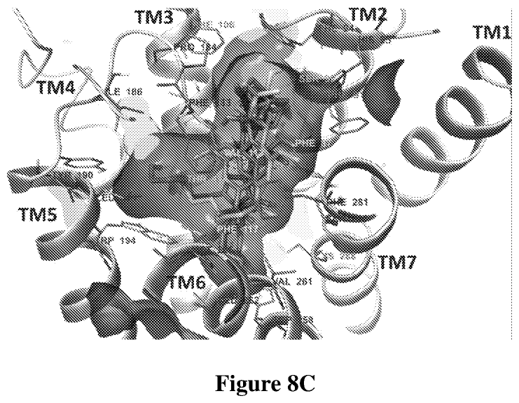

Description of the crystal structures: The crystals of 1b, 1d, 4b and 5d were prepared and determined by single crystal X-ray diffraction. Their crystal data and structure refinement are shown in Table 1. The observed hydrogen bonds are listed in Table 2. The molecular ellipsoids are shown in FIGS. 7 and 8. Compound 1b (FIG. 7A) was recrystallized in MeC(O)Et. There is a symmetric aromatic group on the “C7-C11”. On the other hand, in 4b (FIG. 7B), the fluorinated derivative of 1b, the F on the aromatic ring changed the symmetry.

The bond length of C(15)-H(15) in 1b is 0.9300 A° while that of C(15)-F(1) in 4b is 1.357(4) Å. The bond lengths are within normal ranges (1.09 Å for C—H bond) [20] and 1.35 Å for C—F bond. The C—F bond length is slightly longer than C—H. The dihedral angles of C(16)-C(15)-H(15) and F(1)-C(15)-C(16) are 119.2 and 116.7 respectively. In both compounds, 1b and 4b, the molecules are interlinked by intermolecular hydrogen bonds. Regarding compounds 1d and 5d (FIGS. 8A and 8B), the replacement of a methoxy group with a hydroxyl group on the C16 position of the aromatic ring increased the O(1)-C(7)-C(2) dihedral angle by 4° and decreased the O(1)-C(7)-C(6) dihedral angle by 6°. In the dimethoxy compound, the C7 OH group is making a hydrogen bond with the C12 OMe group. However, when this compound is demethylated, the methoxy group on the C16 carbon is removed and the hydrogen bond now is with the C16 OH group.

TABLE 1
Crystal data and structure refinement for compounds of the invention 1b, 1d, 4b and 5d

| Parameters | Compound 1b | | Compound 4b | | Compound 1d | | Compound 5d | |
|---|---|---|---|---|---|---|---|---|
| Empirical | C₂₃H₃₆O₃ | | C₂₃H₃₅F O₃ | | C₂₇ H₄₄ O₃ | | C₂₆ H₄₂ O₃ | |
| formula | | | | | | | | |
| Formula weight | 360.52 | | 378.51 | | 416.62 | | 402.60 | |
| Temperature [K] | 293 | (1) | 295 | (1) | 293 | (1) | 293 | (1) |
| Wavelength | 0.71073 | | 0.71073 | | 0.71073 | | 0.71073 | |
| Crystal system | Monoclinic | | Orthorhombic | | Orthorhombic | | Orthorhombic | |
| Space group | P2(1)/n | | P2(1)2(1)2(1) | | P2(1)2(1)2(1) | | P2(1)2(1)2(1) | |
| Unit cell | | | | | | | | |
| dimensions | | | | | | | | |
| a [Å] | 9.6585 | (5) | 7.9475 | (5) | 7.9490 | (4) | 7.8307 | (5) |
| b [Å] | 8.9094 | (4) | 12.9307 | (8) | 12.8962 | (6) | 12.4716 | (7) |
| c [Å] | 24.8603 | (1) | 21.046 | (1) | 24.934 | (1) | 25.775 | (2) |
| α [°] | 90.00 | | 90.00 | | 90.00 | | 90.00 | |
| β [°] | 98.589 | (5) | 90.00 | | 90.00 | | 90.00 | |
| γ [°] | 90.00 | | 90.00 | | 90.00 | | 90.00 | |
| Volume [Å³] | 2115.3 | (2) | 2162.8 | (2) | 2556.1 | (2) | 2517.2 | (3) |
| Z | 4 | | 4 | | 4 | | 4 | |
| Density_{caled} | 1.132 | | 1.162 | | 1.083 | | 1.062 | |
| [mg/m³] | | | | | | | | |
| Absorption | 0.073 | | 0.081 | | 0.068 | | 0.067 | |
| coefficient | | | | | | | | |
| [mm⁻¹] | | | | | | | | |
| F(000) | 792 | | 824 | | 920 | | 888 | |
| Crystal size | 0.44 × 0.40 × 0.16 | | 0.54 × 0.11 × 0.09 | | 0.65 × 0.50 × 0.32 | | 0.54 × 0.30 × 0.09 | |
| [mm³] | | | | | | | | |
| Theta range [°] | 4.338 to 53.996 | | 3.87 to 53.996 | | 4.544 to 54 | | 3.628 to 49.984 | |
| for data | | | | | | | | |
| collection | | | | | | | | |
| Index ranges | −12 <= h <= 12 | | −10 <= h <= 10 | | −10 <= h <= 10 | | −9 <= h <= 9 | |
| | −11 <= k <= 11 | | −16 <= k <= 16 | | −16 <= k <= 16 | | −14 <= k <= 14 | |
| | −31 <= 1 <= 31 | | −26 <= 1 <= 26 | | −31 <= 1 <= 31 | | −30 <= 1 <= 30 | |
| Reflections | 21747 | | 22791 | | 27024 | | 22451 | |
| collected | | | | | | | | |
| Independent | 4626 [R(int) = 0.0444] | | 4721 [R(int) = 0.0790] | | 5576 [R(int) = 0.0737] | | 4435 [R(int) = 0.0840] | |
| reflections | | | | | | | | |
| max. and min. | | | | | | | | |
| transmission | | | | | | | | |
| Data/restraints/ | 4626/0/253 | | 4721/0/254 | | 5576/0/283 | | 4435/0/277 | |
| parameters | | | | | | | | |
| Goodness-of-fit | 1.103 | | 1.150 | | 1.072 | | 1.115 | |
| on F² | | | | | | | | |
| Final R indices | 0.0663, 0.1628 | | 0.0679, 0.1204 | | 0.0804, 0.2040 | | 0.0906, 0.1781 | |
| [I > 2σ(I)]; R₁, | | | | | | | | |
| wR₂ | | | | | | | | |
| R₁, wR₂ (all | 0.0817, 0.1722 | | 0.1064, 0.1373 | | 0.0971, 0.2160 | | 0.1359, 0.1969 | |
| data) | | | | | | | | |
| Largest diff. | 0.25/−0.22 | | 0.22/−0.21 | | 0.29/−0.30 | | 0.21/−0.17 | |
| peak and hole | | | | | | | | |
| [e · Å⁻³] | | | | | | | | |

TABLE 2
Hydrogen bonds for compounds of the invention 1b, 1d, 4b and 5d (Å, u)

| | | d(D- | | | |
|---|---|---|---|---|---|
| Compound | D-H . . . A | H) | d(H . . . A) | d(D . . . A) | <(DHA) |
| 1b | O(1)-H(1) . . . O(3) | 0.85(3) | 1.78(3) | 2.531(2) | 147(3) |
| 4b | O(1)-H(1) . . . O(3) | 0.89(5) | 1.81(5) | 2.564(4) | 141(5) |
| 1d | O(1)-H(1) . . . O(3) | 0.87(6) | 1.75(6) | 2.518(5) | 145(5) |
| 5d | O(2)-H(2) . . . O(1) | 0.95(7) | 1.66(7) | 2.483(6) | 142(6) |
| 5d | O(1)-H(1) . . . O(2)#1 | 0.92(7) | 1.93(7) | 2.795(6) | 156(6) |

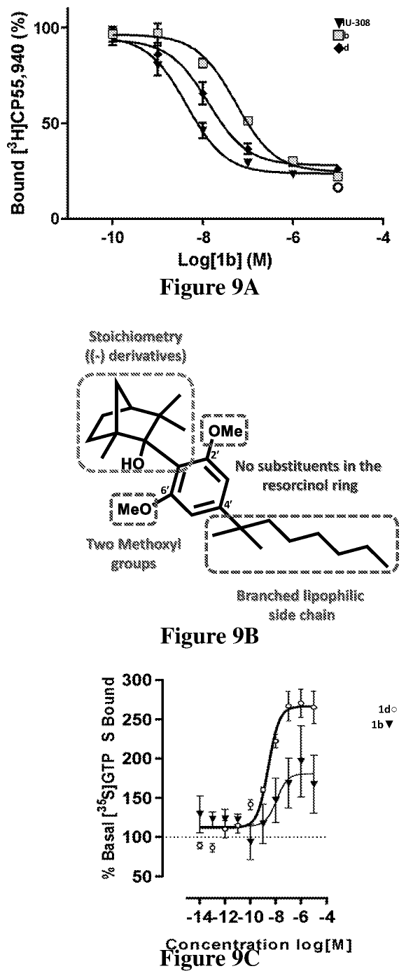

Affinity for Cannabinoid Receptors. The compounds were further characterized using a radioligand binding assay to determine their affinities for CB1R and CB2R based on each test compound's ability to displace the radiolabeled CB1R/CB2R agonist CP-55,940 from membranes prepared from a mouse brain (a source of CB1R) and membranes of cells expressing the human CB2R. Inhibition constant values (Ki) from the respective competition binding curves are listed in Table 3 in which HU-308 was included for comparison. It is worth mentioning that the mouse, rat and human CB1Rs have 97-99% sequence identity across species, and therefore are not expected to exhibit variations in their Kᵢ values; however, the CB2R shows less homology (˜82%) between species and that could cause species-related differences in affinity. Therefore, the compounds were assayed using membranes from CHO-K1 cells expressing the human CB2R (hCB2R). As shown in Table 3, the fenchone derivatives showed high selectivity towards hCB2R over mCB1R except for compounds 1b, 1d and 5d that bind to the mCB1 very weakly (Kᵢ=2288, 427.4 and 451.2 nM respectively). In most compounds, the (−) analogues prepared from the (−)-fenchone (1b, 1d, 1f, 2b, 3b, 3f, 3h, 4b, 4d, 5b and 5d) showed higher affinity towards hCB2R than their (+) counterparts prepared from (+)-fenchone (1a, 1c, 1e, 2a, 3a, 3e, 3g, 4a, 4c, 5a and 5c). It was observed that the affinity for the hCB2R can be optimized by varying the length of the side chain at C4′ for the fenchone-alkylresorcinol dimethyl ether derivatives (1a-f). Thus, the analogue with one methyl group at C4′ (1f) had the least affinity to hCB2R (Kᵢ=233.1 nM) compared to 1b (with a pentyl group) and 1d (with a dimethylheptyl group). Accordingly, 1a (+ isomer) with a pentyl side chain displaced the binding of [³H]CP-55,940 to hCB2R with a Kᵢ value of 47.7 nM and produced no measurable inhibition of [³H]CP-55,940 binding to mCB1R. On the other hand, 1b (− isomer) inhibited [³H]CP-55,940 binding to hCB2R more strongly (Kᵢ=14.45 nM) with no detectable inhibition of [³H]CP-55,940 binding to mCB1R. Other potent compounds carry the dimethylheptyl substituent at C4′, which is typical for synthetic cannabinoids. 1c (+ isomer) and 1d (− isomer) with a dimethylheptyl side chain inhibited binding of [³H]CP-55,940 to hCB2R with a Kᵢ value of 56.81 nM and 3.509 nM, respectively. The compound 1d had a 121-fold selectivity for the hCB2R over the mCB1R. The displacement of [³H]CP-55940 by HU-308, 1b and 1d from specific binding sites in membranes from cells expressing hCB2Rs is shown in FIG. 9A. The change in the position of the chain from the C4′ to C5′ (2a and 2b) reduced the affinity towards hCB2R by an order of magnitude (Kᵢ=223.5 and 73.38 nM respectively). Also, the presence of only one methoxyl group in the aromatic part (3a-h) reduced the affinity to hCB2R dramatically (Table 3).

Introducing a fluorine atom in the aromatic part of 1a-d (4a-d) or demethylating it (5a-d) reduced the binding affinity of 1a-d to the hCB2R by almost one order of magnitude (Table 3). Analysis of the structure-activity relationships (SAR) of all of these analogs revealed several structural features for maintaining CB2R affinity and selectivity, including the stoichiometry of the compounds that should be the (−) derivatives, the presence of a branched lipophilic side chain at C4′, dimethoxy groups in the positions C2′ and C6′ and no substituents in the aromatic ring (FIG. 9B).

TABLE 3
Affinities of compounds of the invention for mCB1 and hCB2 receptors

| | | 95% | | 95% | |
|---|---|---|---|---|---|
| | Kᵢ (nM) | Confidence | Ki (nM) | Confidence | |
| Compound | mCB1R | Interval (nM) | hCB2R | Interval (nM) | mCB1R/hCB2R |
| 1a | >10 μM | — | 47.7 | (21.17-107.5) | — |
| 1b | 2288 | (129.9-40330) | 14.45 | (9.268-22.53) | 158.3 |
| 1c | NB | — | 56.81 | (39.40-81.90) | — |
| 1d | 427.4 | (208.6-875.3) | 3.509 | (2.067-5.958) | 121.8 |
| 1e | NB | — | >10 μM | | — |
| 1f | NB | — | 233.1 | (171.3-317.2) | — |
| 2a | NB | — | 223.5 | (126-396.6) | — |
| 2b | >10 μM | — | 73.38 | (47.99-112.2) | — |
| 3a | NB | — | 1012 | (375.9-2723) | — |
| 3b | NB | — | 610.2 | (44.6-8343) | — |
| 3c | NB | — | 444.1 | (246.5-799.9) | — |
| 3d | NB | — | 834.2 | (414-1681) | — |
| 3e | NB | — | 2874 | (150.4-54940) | — |
| 3f | NB | — | 1449 | (135.1-15540) | — |
| 3g | NB | — | 1651 | (65.36-41720) | — |
| 3h | NB | — | 494.3 | (171.2-1427) | — |
| 4a | >10 μM | — | 155.4 | (13.51-1787) | — |
| 4b | >10 μM | — | 28.33 | (8.314-96.51) | — |
| 4c | NB | — | 438.4 | (246-781.2) | — |
| 4d | NB | — | 56.56 | (30.88-103.6) | — |
| 5a | >10 | — | 4978 | (840.9-29470) | — |
| 5b | >10 | — | 36.53 | (17.03-78.37) | — |
| 5c | NB | — | 107.4 | (55.66-207.2) | — |
| 5d | 451.2 | (184.5-1103) | 24.57 | (16.19-37.29) | 18.4 |
| HU-308 | NB | — | 1.161 | (0.709-1.901) | — |

NB = No binding detected at concentrations up to 10 μM; >10 μM = Displacement of radioactive ligand detected at high concentrations competing ligand.

Functional Characterization. By using the [³S]GTPγS binding assay, the activity (agonism, antagonism, inverse agonism) properties of 9 key compounds was explored, that showed the highest affinity for the hCB2R. Data are listed in Table 4 in which the CB2R agonist HU-308 is included for comparison. The testing results show that most compounds stimulate the GTPγS binding to CB2R, indicating that these compounds behaved as potent agonists at the hCB2R. The (−) compounds with the dimethylheptyl side chains at C4′ (1d, 4d and 5d) were highly efficacious with 1d being more potent (EC₅₀=2.6 nM; E₍ₘₐₓ₎=89.63%) than its monomethoxy (5d) (EC₅₀=14.8 nM; E₍ₘₐₓ₎=105.05%) and its fluorinated analogues (4d) (EC₅₀=104 nM; E₍ₘₐₓ₎=118.7%). The (−) compounds with the pentyl side chain at C4′ (1b, 4b and 5b) were less potent and less efficacious than their dimethylheptyl counterparts (Table 4) (FIG. 9C). Compound 2b with a hexyl side chain in C5′ instead of C4′ weakly stimulated the [35S]GTPγS binding to hCB2R. In the same assay, one of the analogs (1a, the (+) analogue of 1b) with a pentyl side chain weakly reduced the [35S]GTPγS binding in hCB2R membrane (IC₅₀=1581 nM) indicating that it is an inverse agonist.

The most potent compounds in this assay, 1b, 1d and 5d, appeared to be less potent and efficacious than HU-308 at activating the hCB2 receptor in the [35S]GTPγS binding assay (Table 4). However, the mean EC₅₀ that it displayed in this assay was not significantly different from the corresponding EC₅₀ values of HU-308 an indicated by the overlap of the 95% confidence limits. Analysis of the SAR revealed that the structural features requirements for maintaining CB2R agonism are the same as those required for maintaining affinity and selectivity (FIG. 9B).

TABLE 4
Functional potencies (EC₅₀) of key fenchone derivatives
and HU-308 for the hCB2R (±SE confidence limits)

| | | 95% | |
|---|---|---|---|
| Com- | | Confidence | |
| pound | EC₅₀(nM)¹ | Interval (nM) | E(max) %² |
| 1a | 1581 (inverse agonist) | (146.4- . . .) | 24.08 |
| 1b | 11.59 (agonist) | (2.249-70.95) | 65.1 |
| 1c | 121.4 (agonist) | (13.63-676.5) | 87.15 |
| 1d | 2.594 (agonist) | (1.009-6.754) | 89.63 |
| 2b | 1943 (agonist) | (98.02- . . .) | 46.2 |
| 4b | 305.3 (agonist) | (113.5-807.4) | 54.2 |
| 4d | 104.1 (agonist) | (56.24-185.8) | 118.7 |
| 5b | 82.78 (agonist) | (31.93-193.3) | 69.5 |
| 5d | 14.83 (agonist) | (7.8-29.18) | 105.05 |
| HU-308 | 1.008 (agonist) | (0.033-8.945) | 100 |

¹Functional potencies at hCB2R were determined by measuring the increase in [³⁵S]GTPγS binding to the receptor. EC₅₀ values were calculated using
nonlinear regression analysis. Data are the average of two independent experiments run in triplicate.
²E(max) values (maximal effects) is presented as the percentage of HU-308 response at 1 μM.

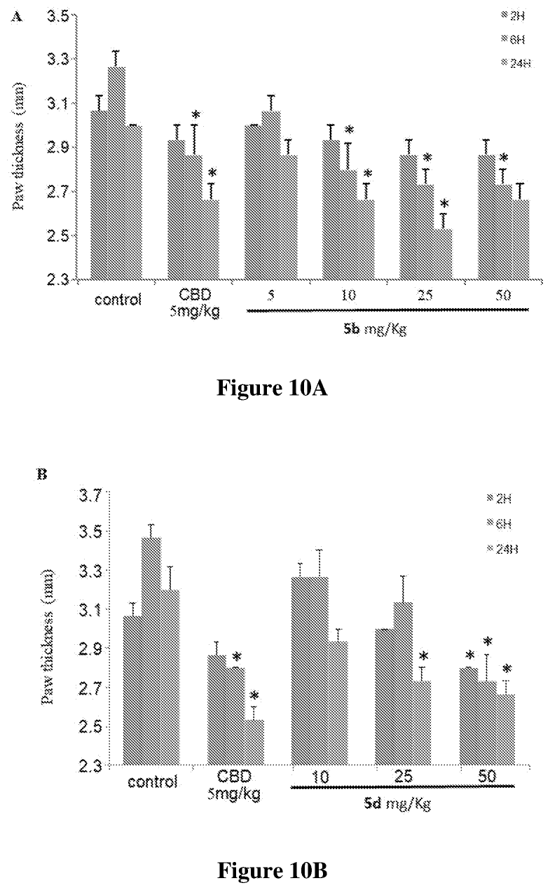

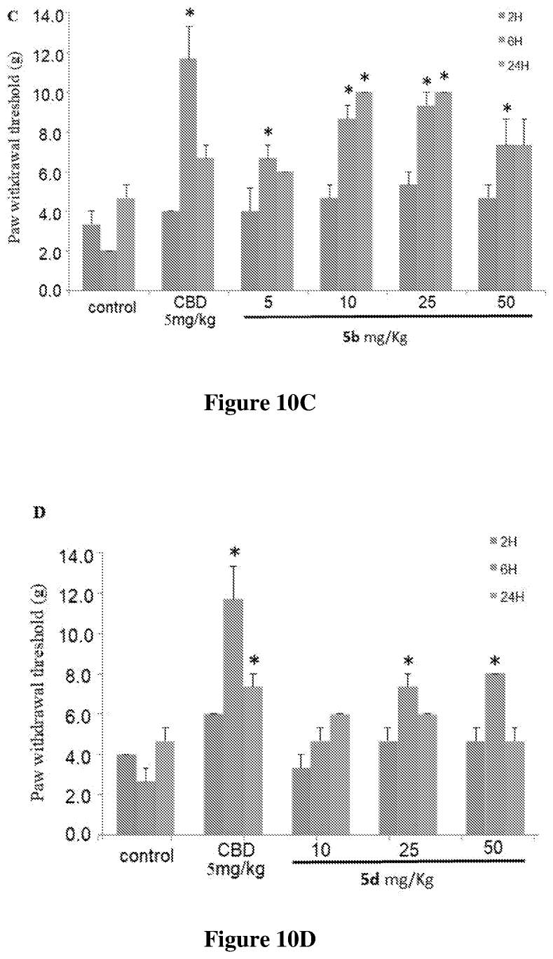

Effect of 5b and 5d on Inflammation and Hyperalgesia (Pain Sensation): In this study the mouse model of zymosan-induced inflammation was used to investigate the anti-inflammatory and anti-nociceptive activities of 5b and 5d. Cannabidiol (CBD), a major constituent of Cannabis, resembles fenchone derivatives in structure. It has powerful anti-inflammatory and anti-anxiety properties and therefore it is used as a positive control. The extent of hind paw swelling was determined 2, 6 and 24 h following paw injection of 60 μg zymosan together with ip of CBD or various amounts of 5b or 5d as indicated in the graphs. The maximum inhibition of inflammation occurred after an injection of 5 mg/kg CBD with 40% and 43% inhibition after 6 and 24 h, respectively (p<0.01) (FIG. 10A). In accordance with these findings, the anti-nociceptive effect, as determined by the von Frey monofilament assay peaked at 6 h (p<0.01) (FIG. 10C). However, when 5b was used, a correlative dose-response was observed with increased inhibition of inflammation upon increased doses of the compound, reaching 40% inhibition for the 10 mg/kg and 50% inhibition at the 25 and 50 mg/kg after 6 h (p<0.005) (FIG. 10A). After 24 h, there was 43% inhibition at the 10 mg/kg and the maximum inhibition was reached at 25 mg/kg with 72% inhibition (p<0.005) (FIG. 10A). These two dosages, 10 and 25 mg/kg of 5b also showed strong anti-nociceptive effects after 6 and 24 h (p<0.005) (FIG. 10C). The compound 5d gave 55% inhibition of inflammation at 25 mg/kg after 24 h and 67% and 56% inhibition at 50 mg/kg after 6 and 24 h respectively (p<0.01) (FIG. 10B). At these two dosages, 25 and 50 mg/kg, 5d also showed moderate anti-nociceptive effects after 6 h (p<0.005) (FIG. 10D).

The anti-inflammatory and anti-nociceptive activities of 5d (with a dimethylheptyl side chain at C4′) was also examined (FIG. 10A-10D). In this experiment, CBD exhibited 58% and 78% inhibition of inflammation after 6 and 24 h, respectively (p<0.01) (FIG. 10B). Also, the anti-nociceptive effect peaked at 6 h (p<0.01) (FIG. 10D). The compound 5d, at a dose of 25 mg induced 56% inhibition of inflammation after 24 h (p<0.01) (FIG. 10B) and at a 50 mg/kg dose, it inhibited inflammation by 38%, 67% and 56% after 2, 6 and 24 h respectively. These two dosages, 25 and 50 mg, also showed moderate anti-nociceptive effects after 6 h (p<0.005) (FIG. 10D).

### Experimental Section

Reagents and solvents were purchased from Biolab LTD (Jerusalem, Israel), J. T. Baker (Center Valley, PA, USA), Sigma-Aldrich (Rehovot, Israel), Acros (Yehud, Israel), Alfa Aesar (Lancashire, UK) and Merck (Darmstadt, Germany) and were used without further purification.

NMR spectra were recorded at 500 MHz (2D ¹H and ¹³C NMR) and 300 MHz (¹H, ¹³C and ¹⁹F NMR) using deuterated chloroform (CDCl₃, δ=7.26 ppm) with tetramethylsilane (TMS) as internal standard. Thin-layer chromatography (TLC) was run on silica gel 60F₂₅₄ plates (Merck). Column chromatography was performed on silica gel 60 Å (Merck). Compounds were located using a UV lamp at 254 nm. GCMS analyses were performed on an HP GCMS instrument (Model GCD PLUS) with an EI detector and 30 m methyl silicone column. Optical rotations were measured on polarimeter (Optical Activity) in a 2.00 dm cell and 25° C.

### Synthesis:

### Methylation of Alkyl Phenols/Resorcinols:

Methyl iodide (12 mmol) was added to a solution of alkyl phenol/resorcinol (1.51 mmol) and K₂CO₃ (12 mmol) in dry DMF (5 mL). After stirring at room temperature for 24 h, the mixture was diluted with water (40 ml) and extracted with ether. The organic layer was washed with water, dried, and evaporated followed by purification by column chromatography on silica gel with ether/petroleum ether (2-4%).

### Synthesis of 2-(2,6′-dimethoxy-4′-pentylphenyl)-1,3,3-trimethylbicyclo[2.2.1]heptan-2-ol (1a & 1b)

To 0.5 g (2.4 mmol) 1,3-dimethoxy-5-pentylbenzene in 5 ml dry THF at room temperature was added n-BuLi (1.6 M in hexane, 3.3 ml, 5.28 mmol) and the resulting solution was refluxed under N₂ for 2.5 h, cooled to r.t and the ketone (fenchone, 2.64 mmol, 0.401 g) in 1 ml THF added. The reaction mixture was refluxed for 3 h and then for 18 h at r.t. The reaction was worked up by the addition of saturated NH₄Cl solution and extracted with ether. After washing (H₂O) and drying (MgSO₄), the solvent was evaporated to give the crude target compound, which was purified by chromatography, generally using with ether/petroleum ether (3-5%) (yield 30%). HPLC purity: 98.6%. ¹H-NMR (500 MHz, CDCl₃): 6.42 (s, 1H, H-3), 6.40 (s, 1H, H-5′), 3.79 (s, 3H, H-7′), 3.87 (s, 3H, H-8′), 2.57 (t, 2H, H-1″), 1.62 (m, 2H, H-2″), 1.37 (m, 4H, H-3″,4″), 0.93 (t, 3H, H-5″), 0.62 (s, 3H, H-7), 2.75 (dd, 1H, H-8), 1.12 (m, 5H, H-8, H-9, H-6), 1.17 (m, 4H, H-10, H-5), 2.41 (m, 1H, H-6), 1.71 (m, 2H, H-5, H-4). ¹³C-NMR (500 MHz, CDCl₃): 159.16 (C-2′), 156.51 (C-6′), 142.23 (C-4′), 120.71 (C-1′), 105.59 (C-3′), 104.85 (C-5′), 55.57 (C-7′), 54.32 (C-8′), 35.91 (C-1″), 30.80 (C-2″), 31.64 (C-3″), 22.00 (C-4″), 14.07 (C-5″), 54.23 (C-1), 87.95 (C-2), 46.52 (C-3), 50.78 (C-4), 23.97 (C-5), 35.13 (C-6), 41.16 (C-8), 28.48 (C-7), 18.42 (C-10), 22.05 (C-9). LC-UV-MS (ESI): m/z calculated for C₂₃H₃₆O₃ 360.27, found 343.09 (m-OH) for 1a and 342.98 (m-OH) for 1b. [α]_{D}²⁵+112.7° for 1a and −106.6° for 1b.

### Synthesis of 2-(2,6′-dimethoxy-4′-(2″-methyloctan-2″-yl)phenyl)-1,3,3-trimethyl-bicyclo[2.2.1]heptan-2-ol (1c & 1d)

To a solution of the 1,3-dimethoxy-5-(2-methyloctan-2-yl)benzene (1 g, 3.8 mmol) in 32 ml of dry THF was added a 1.6 M solution of n-BuLi in hexane (8.8 mmol, 5.4 ml) at 0° C. with stirring under N₂. After additional stirring for 1 h at 0° C., a solution of the fenchone (6 mmol, 0.9 g) in 1 ml of dry THF was added all at once. The reaction mixture was stirred for 0.5 h at 0° C. and then for 18 h at r.t. The reaction was worked up by the addition of saturated NH₄Cl solution and extracted with ether. After washing (H₂O) and drying (MgSO₄), the solvent was evaporated to give the crude target compound, which was purified by chromatography with ether/petroleum ether (3-4%) (yield 62.4%). HPLC purity: 98.9%. ¹H-NMR (500 MHz, CDCl₃): 6.50 (s, 1H, H-3′), 6.48 (s, 1H, H-5′), 3.86 (s, 3H, H-7′), 3.77 (s, 3H, H-8′), 1.55 (m, 2H, H-3″), 1.26 (m, 10H, H-4″,5″,1″,9″), 11.20 (m, 4H, H-6″,7″), 0.84 (t, 3H, H-8″), 0.58 (s, 3H, H-7), 2.73 (dd, 1H, H-8), 1.07 (m, 5H, H-8, H-9, H-6), 1.15 (m, 3H, H-10), 1.35 (m, 1H, H-5) 2.38 (m, 1H, H-6), 1.68 (m, 2H, H-5, H-4). ¹³C-NMR (500 MHz, CDCl₃): 158.90 (C-2′), 156.21 (C-6′), 149.144 (C-4′), 120.30 (C-1′), 103.33 (C-3′), 102.61 (C-5′), 55.52 (C-7′), 54.28 (C-8′), 37.74 (C-2″), 44.41 (C-3″), 22.62 (C-4″), 29.94 (C-5″), 31.70 (C-6″), 29.94 (C-7″), 14.07 (C-8″), 28.75 (C-1″), 28.75 (C-9″), 54.16 (C-1), 87.87 (C-2), 46.54 (C-3), 50.73 (C-4), 23.96 (C-5), 35.07 (C-6), 28.42 (C-7), 41.17 (C-8), 18.48 (C-9), 22.00 (C-10). LC-UV-MS (ESI): m/z calculated for C₂₇H₄₄O₃ 416.33, found 399.12 (m-OH) for 1c and 399.02 (m-OH) for 1d. [α]_{D}²⁵+96.88° for 1c and −95.95° for 1d.

### Synthesis of 2-(2,6′-dimethoxy-4′-methylphenyl)-1,3,3-trimethylbicyclo[2.2.1]-heptan-2-ol (1e & 1f)

Same procedure as above but 1,3-dimethoxy-5-methylbenzene was used and no reflux was done (yield 22%). HPLC purity: 98.4%. ¹H-NMR (300 MHz, CDCl₃): 6.42 (s, 1H, H-3′), 6.40 (s, 1H, H-5′), 3.76 (s, 3H, H-7′), 3.85 (s, 3H, H-8′), 2.38 (t, 3H, H-1″), 0.60 (s, 3H, H-7), 2.72 (dd, 1H, H-8), 1.10 (m, 9H, H-8, H-9, H-10, H-6), 1.36 (m, 1H, H-5), 2.30 (m, 1H, H-6), 1.66 (m, 2H, H-5, H-4). LC-UV-MS (ESI): m/z calculated for C₁₉H₂₈O₃ 304.20, found 287.05 (m-OH) for 1e and 1f. [α]_{D}²⁵+91.5° for 1e and −82.90° for 1f.

### Synthesis of 2-(3′-hexyl-2,6′-dimethoxyphenyl)-1,3,3-trimethylbicyclo[2.2.1]heptan-2-ol (2a & 2b)

Same procedure as above but 1-hexyl-2,4-dimethoxybenzene was used and no reflux was done (yield 28%). HPLC purity: 98.6%. ¹H-NMR (300 MHz, CDCl₃): 7.06 (d, 1H, H-4′), 6.61 (d, 1H, H-5′), 3.81 (s, 3H, H-7′), 3.75 (s, 3H, H-8′), 2.57 (t, 2H, H-1″), 1.64 (m, 2H, H-2″), 1.51 (m, 2H, H-3″), 1.33 (m, 4H, H-4″,5″), 0.89 (t, 3H, H-6″), 0.73 (s, 3H, H-7), 2.82 (dd, 1H, H-8), 1.05 (m, 5H, H-8, H-9, H-6), 1.33 (m, 4H, H-10, H-5), 2.36 (m, 1H, H-6), 1.73 (m, 2H, H-5, H-4). LC-UV-MS (ESI): m/z calculated for C₂₄H₃₈O₃ 374.28, found 357.08 (m-OH) for 2a and 2b. [α]_{D}²⁵+84.82° for 2a and −90.32° for 2b.

### Synthesis of 2-(2′-methoxy-5′-propylphenyl)-1,3,3-trimethylbicyclo[2.2.1]heptan-2-ol (3a & 3b)

Same procedure as for 1a & 1b but 1-methoxy-4-propylbenzene was used (yield 53%). HPLC purity: 98.8%. ¹H-NMR (CDCl₃): ¹H-NMR (300 MHz, CDCl₃): 7.33 (s, 1H, H-6′), 6.80 (d, 1H, H-3′), 7.00 (d, 1H, H-4′), 3.86 (s, 3H, H-7′), 2.54 (m, 2H, H-1″), 1.57 (m, 2H, H-2″), 0.93 (t, 3H, H-3″), 0.46 (s, 3H, H-7), 2.54 (m, 1H, H-8), 1.12 (m, 5H, H-8, H-9, H-6), 1.30 (m, 4H, H-10, H-5), 2.23 (m, 1H, H-6), 1.73 (m, 2H, H-5, H-4). LC-UV-MS (ESI): m/z calculated for C₂₀H₃₀O₂ 302.22, found 285.07 (m-OH) for 3a and 3b. [α]_{D}²⁵+63.44° for 3a and −62.96° for 3b.

### Synthesis of 2-(2′-methoxy-5′-pentylphenyl)-1,3,3-trimethylbicyclo[2.2.1]heptan-2-ol (3c & 3d)

Same procedure as for 1a & 1b but 1-methoxy-4-pentylbenzene was used (yield 25%). HPLC purity: 99.3%. ¹H-NMR (300 MHz, CDCl₃): 7.31 (s, 1H, H-6′), 6.77 (d, 1H, H-3′), 6.94 (d, 1H, H-4′), 3.81 (s, 3H, H-7′), 2.52 (m, 2H, H-1″), 1.57 (m, 2H, H-2″), 1.39 (m, 4H, H-3″,4″), 0.89 (t, 3H, H-5″), 0.45 (s, 3H, H-7), 2.54 (m, 1H, H-8), 1.17 (m, 5H, H-8, H-9, H-6), 1.29 (m, 4H, H-10, H-5), 2.24 (m, 1H, H-6), 1.72 (m, 2H, H-5, H-4). LC-UV-MS (ESI): m/z calculated for C₂₂H₃₄O₂ 330.26, found 313.09 (m-OH) for 3c and 3d. [α]_{D}²⁵+99.290 for 3c and −96.99° for 3d.

### Synthesis of 2-(2′-methoxy-5′-(tert-pentyl)phenyl)-1,3,3-trimethylbicyclo[2.2.1]heptan-2-ol (3e & 3f)

Same procedure as for 1a & 1b but 1-methoxy-(4-tert-pentyl)benzene was used (yield 44%). HPLC purity: 99.5%. ¹H-NMR (300 MHz, CDCl₃): 7.48 (s, 1H, H-6′), 6.81 (d, 1H, H-3′), 7.09 (d, 1H, H-4′), 3.86 (s, 3H, H-7′), 1.62 (m, 2H, H-3″), 0.66 (t, 3H, H-4″), 0.43 (s, 3H, H-7), 2.48 (m, 1H, H-8), 1.16 (m, 5H, H-8, H-9, H-6), 1.36 (m, 4H, H-10, H-5), 2.22 (m, 1H, H-6), 1.73 (m, 2H, H-5, H-4). LC-UV-MS (ESI): m/z calculated for C₂₂H₃₄O₂ 330.26, found 313.09 (m-OH) for 3e and 313.10 (m-OH) for 3f. [α]_{D}²⁵+81.070 for 3e and −82.68° for 3f.

### Synthesis of 2-(2′-methoxy-5′-octylphenyl)-1,3,3-trimethylbicyclo[2.2.1]heptan-2-ol (3g & 3h)

Same procedure as for 1a & 1b but 1-methoxy-4-(2-methylheptan-2-yl)benzene was used (yield 20%). HPLC purity: 95.5%. ¹H-NMR (300 MHz, CDCl₃): 7.52 (s, 1H, H-6′), 6.78 (d, 1H, H-3′), 7.13 (d, 1H, H-4′), 3.86 (s, 3H, H-7′), 2.64 (m, 2H, H-1″), 1.70 (m, 2H, H-2″), 1.43 (m, 10H, H-3″,4″,5″,6″,7″), 0.73 (t, 3H, H-8″), 0.44 (s, 3H, H-7), 2.45 (m, 1H, H-8), 1.12 (m, 5H, H-8, H-9, H-6), 1.34 (m, 4H, H-10, H-5), 2.23 (m, 1H, H-6), 1.70 (m, 2H, H-5, H-4). LC-UV-MS (ESI): m/z calculated for C₂₅H₄₀O₂ 372.30, found 355.12 (m-OH) for 3g and 3h. [α]_{D}²⁵ 88.52° for 3g and 81.82° for 3h.

Fluorination with Selectflour®:

0.48 mmol of Selectflour® was dissolved in 2.6 ml acetonitrile. The solution was cooled to 0-5° C. and the fenchone-dimethoxyalkylresorcinol (0.48 mmol) in 2.6 ml acetonitrile was added to it. The reaction was stirred at this temperature for 1.5 h, and then the reaction was left stirring overnight at room temperature. Ether was added to the reaction mixture and then the mixture was washed with brine. Removal of solvent afforded the desired products, which were purified by chromatography with ether/petroleum ether (0-2%).

### 2-(3′-fluoro-2′,6′-dimethoxy-4′-pentylphenyl)-1,3,3-trimethylbicyclo[2.2.1]hept-an-2-ol (4a & 4b)

(yield 17%). HPLC purity: 97.3%. ¹H-NMR (300 MHz, CDCl₃): 6.37 (s, 1H, H-5′), 3.92 (s, 3H, H-8′), 3.97 (s, 3H, H-7′), 2.55 (t, 2H, H-1″), 1.66 (m, 2H, H-2″), 1.32 (m, 4H, H-3″,4″), 0.88 (t, 3H, H-5″), 0.61 (s, 3H, H-7), 2.71 (dd, 1H, H-8), 1.12 (m, 5H, H-8, H-9, H-6), 1.16 (m, 4H, H-10, H-5), 2.48 (m, 1H, H-6), 1.58 (m, 2H, H-5, H-4) ¹⁹F-NMR (300 MHz, CDCl₃): −140 LC-UV-MS (ESI): m/z calculated for C₂₃H₃₅FO₃ 378.26, found 361.09 (m-OH) for 4a and 360.91 (m-OH) for 4b, [α]_{D}²⁵ 110.01° for 4a and 103.13° for 4b.

### 2-(3′-fluoro-2,6′-dimethoxy-4′-(2″-methyloctan-2″-yl)phenyl)-1,3,3-trimethyl-bicyclo[2.2.1]heptan-2-ol (4c & 4d)

(yield 22%). HPLC purity: 90%. ¹H-NMR (300 MHz, CDCl₃): 6.41 (s, 1H, H-5′), 3.96 (s, 3H, H-7′), 3.90 (s, 3H, H-8′), 1.57 (m, 2H, H-3″), 1.26 (m, 10H, H-4″,5″,1″,9″), 1.21 (m, 4H, H-6″,7″), 0.84 (t, 3H, H-8″), 0.61 (s, 3H, H-7), 2.75 (dd, 1H, H-8), 1.08 (m, 5H, H-8, H-9, H-6), 1.15 (m, 3H, H-10), 1.35 (m, 1H, H-5), 2.40 (m, 1H, H-6), 1.72 (m, 2H, H-5, H-4). ¹⁹F-NMR (CDCl₃): −134.6 LC-UV-MS (ESI): m/z calculated for C₂₇H₄₃FO₃ 434.32, found 417.13 (m-OH) for 4c and 417.12 (m-OH) for 4d, [α]_{D}²⁵ 69.98° for 4c and 58.30° for 4d.

Demethylation with Sodium Ethanethiolate/DMF.

4-8 ml (2-4 mmol) of a 0.5 M solution of NaSEt in DMF was added to the aromatic methoxy compound (1 mmol) and the resulting solution was heated in an oil bath at 115-120° C. under N₂. The completion of the reaction in each case was determined by TLC. The cooled reaction mixture was then acidified with 10% aqueous HCl and extracted with EtOAc (3×10 ml). The combined organic extracts were washed with 10% aqueous NaOH (3×3 ml) and H₂O (3 ml) and dried (MgSO₄). Removal of solvent afforded the desired products, which were purified by chromatography with ether/petroleum ether (6-8%).

### 2-(2′-hydroxy-6′-methoxy-4′-pentylphenyl)-1,3,3-trimethylbicyclo[2.2.1]heptan-2-ol (5a & 5b)

(yield 13%). HPLC purity: 98.7%. ¹H-NMR (500 MHz, CDCl₃): 6.38 (s, 1H, H-3′), 6.40 (d, 1H, H-5′), 3.77 (s, 3H, H-7′), 2.51 (t, 2H, H-1″), 1.63 (m, 2H, H-2″), 1.36 (m, 4H, H-3″,4″), 0.92 (t, 3H, H-5″), 0.71 (s, 3H, H-7), 2.82 (dd, 1H, H-8), 1.18 (m, 5H, H-8, H-9, H-6), 1.20 (s, 4H, H-10, H-5), 2.41 (m, 1H, H-6), 2.05 (m, 1H, H-6), 1.74 (m, 2H, H-5, H-4). ¹³C-NMR (500 MHz, CDCl₃): 159.42 (C-6′), 156.40 (C-2′), 143.58 (C-4′), 115.00 (C-1′), 110.96 (C-5′), 102.85 (C-3′), 54.32 (7′), 35.54 (C-1″), 30.52 (C-2″), 31.69 (C-3″), 22.55 (C-4″), 14.06 (C-5″), 55.05 (C-1), 90.86 (C-2), 47.55 (C-3), 50.03 (C-4), 23.30 (C-5), 35.23 (C-6), 41.33 (C-8), 27.90 (C-7), 17.61 (C-10), 22.00 (C-9). LC-UV-MS (ESI): m/z calculated for C₂₂H₃₄O₃ 346.25, found 328.99 (m-OH) for 5a and 328.94 (m-OH) for 5b, [α]_{D}²⁵ 56.25° for 5a and 48.01° for 5b

### 2-(2′-hydroxy-6′-methoxy-4′-(2″-methyloctan-2″-yl)phenyl)-1,3,3-trimethyl-bicyclo[2.2.1]heptan-2-ol (5c & 5d)

(yield 12%). HPLC purity: 90%. ¹H-NMR (300 MHz, CDCl₃): 6.50 (s, 1H, H-5′), 6.48 (s, 1H, H-3′), 3.86 (s, 3H, H-7′), 1.55 (m, 2H, H-3″), 1.26 (m, 10H, H-4″,5″,1″,9″), 1.20 (m, 4H, H-6″,7″), 0.84 (t, 3H, H-8″), 0.58 (s, 3H, H-7), 2.73 (dd, 1H, H-8), 1.07 (m, 5H, H-8, H-9, H-6), 1.15 (m, 3H, H-10), 1.35 (m, 1H, H-5), 2.38 (m, 1H, H-6), 1.68 (m, 2H, H-5, H-4). ¹³C-NMR (300 MHz, CDCl₃): 158.90 (C-6″), 156.21 (C-2″), 149.144 (C-4″), 120.30 (C-5″), 118.2 (C-1″), 103.33 (C-3″), 55.52 (C-7″), 37.74 (C-2″), 44.41 (C-3″), 22.62 (C-4″), 29.94 (C-5″), 31.70 (C-6″), 29.94 (C-7″), 14.07 (C-8″), 28.75 (C-1″), 28.75 (C-9″), 54.16 (C-1), 87.87 (C-2), 46.54 (C-3), 50.73 (C-4), 23.96 (C-5), 35.07 (C-6), 28.42 (C-7), 41.17 (C-8), 18.48 (C-9), 22.00 (C-10). LC-UV-MS (ESI): m/z calculated for C₂₆H₄₂O₃ 402.31, found 385.13 (m-OH) for 5c and 384.97 (m-OH) for 5d. [α]_{D}²⁵+96.88° for 1c and −95.95° for 1d.

### X-Ray Crystallography:

A single crystal of the compound was attached to a 400/50 MicroMeshes™ with NVH Oil and transferred to a Bruker SMART APEX CCD X-ray diffractometer equipped with a graphite-monochromator. The system was controlled by a pentium-based PC running the SMART software package. Data were collected at room temperature using MoKα radiation (λ=0.71073 Π). Data processing vas perform using CrysAlisPro, using Olex2, the structure was solved with the SHELXT structure solution program using Intrinsic Phasing and refined with the SHELXL refinement package using Least Squares minimization.

### Binding Assays:

Binding to the CB1R was assessed in a competition displacement assays using [³H]CP-55,940 as the radioligand and crude membranes from mouse brain for the CB1R, as reported previously. Membranes from cells expressing human CB2R were purchased from Charles River, (Cat #A308; Ohio, US). Solutions of test compounds ranging from 0.1 nM to 10 mM were prepared in DMSO. The desired amount of membrane preparation was diluted with ice-cold assay buffer (50 mM Tris-HCl, 2.5 mM EDTA, 5 mM MgCl₂, 0.1% BSA, pH 7.4) and was vortexed. 100 μL of compound was distributed into each tube, followed by addition of 800 μL of diluted membranes (1 μg/tube) and kept on ice until the addition of [³H]CP-55,940. [³H]CP-55,940 was diluted with cold (unlabeled) assay buffer and 100 μL was added into each tube. The assays were incubated for 90 minutes at 30° C. and then immediately filtered on WHATMAN GF/B FilterPaper (Fired) using a Brandel M-24R Harvester followed by six washes with ice cold wash buffer (50 mM Tris-HCl, 2.5 mM EDTA, 5 mM MgCl₂, 0.1% BSA, pH 7.4). Radioactivity was detected by adding the FilterPaper directly to the ULTIMA GOLD scintillation cocktail (PerkinElmer), incubation at 20° C. for 60 min and then counted using a Tri-Carb 4910TR liquid scintillation counter.

### [³⁵S]GTPγS Binding Assay:

The method used for measuring agonist-stimulated binding of [³⁵S]GTPγS was based on a described protocol. The assays were carried out with GTPγS assay buffer (50 mM Tris HCl (pH 7.4), 0.2 mM EGTA, 9 mM MgCl₂, 150 mM NaCl, 1 mg/ml BSA). Briefly, membranes (5 μg protein) were incubated in assay buffer containing 100 μM GDP, 0.05 nM [³⁵S]GTPγS, test compounds at various concentrations in siliconized glass tubes. Bound ligand was separated from free ligand by vacuum filtration. Nonspecific binding was determined using 10 μM GTPS. Basal binding was assayed in the absence of the ligand and in the presence of GDP.

### Animals:

6-8-week old female Sabra mice (Envigo, Israel) were maintained in the SPF unit of the Hebrew University Hadassah Medical School, Jerusalem, Israel. The experimental protocols were approved by the Animal Care Ethical Committee of the Hebrew University-Hadassah Medical School, Jerusalem, Israel. The animals were maintained on standard pellet diet and water ad libitum. The animals were maintained at a constant temperature (20° C.-21° C.) and a 12 h light/dark cycle.

Induction of Paw Inflammation in Mice and Treatment with Fenchone Derivatives:

To induce inflammation, 40 μL of 1.5% (w/v) zymosan A (Sigma) suspended in 0.9% saline was injected into the sub-planter surface of the right hind paw of the mice. Immediately after zymosan injection, fenchone derivative was injected intraperitoneally (i.p.) after the compounds were dissolved in 0.1 mL vehicle containing ethanol:Cremophore:saline at a ratio of 1:1:18. Control mice were injected with the vehicle only. After 2, 6 and 24 hrs, paw swelling and pain perception were measured.

### Measurement of Oedema Formation:

The paw swelling (thickness) was measured by calibrated calipers (0.01 mm), 2, 6 and 24 hrs following injections of zymosan alone or Fenchone derivatives.

### Pain Assay:

The hyperalgesia was evaluated by the paw withdrawal von Frey test at 2, 6, and 24 hrs following injections of zymosan and/or the test compounds. In the von Frey nociceptive filament assay, von Frey calibrated monofilament hairs of logarithmically incremental stiffness (0.008-300 g corresponding to 1.65-6.65 log of force). In this study, only 1.4-60 g corresponding to 4.17 to 5.88 log of force was used, to test the mouse sensitivity to a mechanical stimulus on the swollen paw. The measurements were performed in a quiet room. Before paw pain measurements, the animals were held for 10 sec. The trained investigator applied the filament to the central area of the hind paw with gradual increasing size. The test consisted of poking the middle of the hind paw to provoke a flexion reflex followed by a clear flinch response after paw withdrawal. Each one of the von Frey filaments was applied for approximately 3-4 s to induce the end-point reflex. The first testing was done by using the force filament of 1.4 g. If there was no withdrawal response, the next higher stimulus was tried. The mechanical threshold force (in grams (g)) was defined as the lowest force imposed by two von Frey monofilaments of various sizes, required to produce a paw retraction. The untreated left hind paw served as a control.

### Molecular Modeling.

Molecular-modeling studies were carried out to understand the binding interactions of 1d within the CB2 binding site and compare with the parent CB2 compounds, HU-308 (+) and its (−) enantiomer, HU-433. For the better understanding of interactions and orientations of ligands in the orthosteric binding pocket, all best docked pose of compounds were superimposed and colored differently (FIG. 8C). The electrostatic potential surface shows the hydrophobic nature of the orthosteric site. The enantiomers HU-308 (+) and HU-433 (−) have adopted quite different poses in the binding site of CB2 receptor. The HU-308 has taken constrained binding pose while HU-433 has acquired the extending conformation. 1d has the similar stereochemistry of HU-433 and therefore it has adopted the extended conformation like HU-433 in docking calculation. The interaction of CB2 with 1d is mainly from the hydrophobic and aromatic residues of ECL2, TM2, TM3, TM4, TM5, TM6 and TM7. The bicyclic ring of 1d establishes the extensive hydrophobic interaction network with the residues of extracellular side of the pocket i.e. F912.61, F942.64, F952.65, F1063.25 and 11103.29 while 1,1-dimethylheptyl chain extends towards deep pocket and forms hydrophobic interactions with the residues F1173.36, W1945.53, W2586.48, V2616.51, L2626.52 and F2817.35. The affinity further enhanced by the cooperative 71-71 interaction between the 2,6-dimethoxy phenyl ring of 1d and F872.57 and F183ECL2.

### Measurement of TNFα:

Blood was collected 24 h after zymosan injection, and the sera were assayed for TNFα using a mouse TNFα ELISA kit (R&D Systems, Minneapolis, NN, USA), according to the manufacturer's instructions.

### Molecular Modelling Studies:

The three-dimensional structure of human CB2 (PDB ID: 5ZTY) was downloaded from the protein databank. The missing residues between 222 to 235 were modelled and mutations have been reverted to wildtype residues. The protonation states of all acidic and basic residues were assigned at physiological pH 7.2. The retrained minimization considering 0.30 Å root mean square deviation (RMSD) of all atoms was performed using optimized potentials for liquid simulations extended (OPLS3e) force field. All docking calculations have been performed using two different docking protocols, Schrodinger suit 2020.3 [33] and Autodock 4.5.7. The orthosteric ligand binding site was defined by generating 20 Å grid around the co-crystallized small molecule (AM10257) in Glide whereas 60×60×60 grid points with a 0.375 Å spacing around centroid of AM10257 was generated in Autodock.

The compounds HU-308, HU-433, AM10257 and 1d were prepared at pH 7.0±2.0 using LigPrep module. The docking calculations were performed using the default protocol of GLIDE module. The 10 conformations of each compound were generated using Standard precision (SP) docking. The 10 poses of each conformation were generated using Extra precision (XP) docking. The selection of the best pose was made on the basis of lowest energy and interaction with the active site residues. In Autodock, Lamarckian Genetic Algorithm was used to identified binding poses of each compound. The receptor was kept rigid, whereas the ligand was allowed torsional flexibility. The default parameters were set, but with 2.5×107 energy evaluations. The 50 poses of each compound were generated using Lamarckian Genetic Algorithm. The resulting poses were clustered into groups of 2.0 Å root-mean-square deviation (rmsd). The best scoring pose from the group having a greater number of conformers was chosen as final pose. The software have shown almost similar lowest energy poses of ligands.

### Statistical Analyses:

Statistical analysis was performed with GraphPad Prism software. Statistical analysis details are listed under each figure. The results are presented as value ±SE (standard error). In rare cases where all the measurements give the same values, no SE bar is presented, as no error can be measured. *p<0.05 comparing to control group. #p-value <0.05 in the indicated comparison.

### Fenchone Derivative 1D Effectively Abrogate Joint Damage Following Post-Traumatic OA Lewis Rats:

### Materials and Reagents:

HU308, Fenchones 1D, 1B were synthetized and characterized as shown above and prepared fresh before use, in a solvent comprised of ethanol, cremophor (Kolliphor EL; MI, St Louis; Sigma-Aldrich) and saline at 1:1:18 ratio, respectively. Initially, the required amount of HU308 dissolved in ethanol followed by addition of chromophore with vigorous mechanical agitation to form viscous micelle solution. Finally, the micelle solution was diluted in ice cold saline for injection or culture assays. Vehicle solution consisted of ethanol, cremophor and saline at 1:1:18 ratio. For intra-articular (IA) dosing 50 μL was injected into the tibiofemoral joint, at two doses; 8 and 24 μg/mL. Vehicle control contained 1:1:18 ethanol/Cremophor/saline, while positive control was of IA administered 60 μg/mL FGF-18 (R&D Systems, catalogue No. 8988-F18-050, lot No. BVE0521071) for IA dosing at 50 μL/knee. Notably, all animal procedures detailed below, were carried by Inotiv Boulder, according to the detailed procedures, below.

Agonist and Antagonist cAMP Secondary Messenger Assays:

Cannabinoid receptors belong to G-protein-coupled receptors which may transduce intracellular agonist-antagonist responses regulating in part levels of cAMP within the cells. Fenchone derivates were assayed using Eurofins proprietary assays (i.e. CB1-Antagonist Catalog #86-0007P-2277AN; CB2-Antagonist Catalog ref. 86-0007P-2818AN; CB1-Agonist; Catalog #86-0007P-2277AG; CB2-Agonist-Catalog #86-0007P-2818AG).

Briefly, for antagonist activity, cAMP Hunter™ Gi cell lines overexpress naturally Gi coupled, wild type GPCRs and are designed to detect inhibition of intracellular cAMP production in response to agonist stimulation of the receptor. These cell lines are designed to be used in conjunction with the HitHunter® cAMP Assay Detection Kit.

Similarly, for agonist activity, cAMP Hunter™ Gs cell lines overexpress naturally Gs coupled, wild type GPCRs and are designed to detect increases in intracellular cAMP levels in response to agonist stimulation of the receptor. These cell lines are designed to be used in conjunction with the HitHunter® cAMP Assay Detection Kit. For both assays, human CB1 or CB2 receptors were stably transfected CHO-K1 cells and subjected to positive controls, known to activate the increase of cAMP for both receptors (Agonist control 20 μM Forskolin for CB1 and 25 μM Forskolin for CB2) or inhibit cAMP intracellular levels of CB1 (0.00054 M CP55940) or CB2 (0.0012 M CP55940). Cells were seeded in a total volume of 20 μL into white walled, 384-well microplates and incubated at 37° C. overnight. Prior to testing cell plating media was exchanged with 10 uL of Assay buffer (HBSS+10 mM HEPES). Five 5 μL of agonist (test sample, vehicle or positive control) were added to cells and incubated at 37° C. for 30 minutes. Final assay vehicle concentration was 1% and the results are expressed in relation to the control ligand.

### Determining EC50 in Chondrocyte Cell Cultures:

All human cell cultures were obtained from total knee replacement surgery (TKA) in accordance with Hadassah Medical Center Institutional Review Board approval and in accordance with the Helsinki Declaration ethical principles for medical research involving human subjects (Study #0488-09). Following written informed consent, articular cartilage was obtained from the knee joints of OA patients undergoing total knee arthroplasty (n=51, mean age 71 years, mean body mass index 31 kg/m2, Kellgren and Lawrence score ranging 3-4). Articular cartilage tissue was dissected, chondrocytes isolated and plated as described by Bar Oz et al., (10). Isolated chondrocytes were passaged to passage 3, and plated in 96 well plates with DMEM media containing 10% FCS, 1% Penicillin-streptomycin, 1% Amphotericin B. Cultures were maintained in standard incubation conditions (37° C., 5% CO2) until confluence. All reagents for cell culture were purchased for Biological Industries (Israel, Beit-Haemek Kibutz), unless otherwise indicated. After confluence, chondrocytes were treated with 100 mM Forskolin (positive control, Sigma Aldrich, St Louis) and untreated cells for 45 min until inducing the reaction with the cAMP-Glo™ Assay, according to manufacturer's instructions (Promega, Cat #V1501). Luminescence was measured and a standard curve was simultaneously run per plate. Fenchones 1D,1B and HU308 were measured in 10⁻⁵-10⁻¹² Molar concentration ranges. ΔRLU was calculated by subtracting RLU of untreated sample with RLU of treated sample. Using this ΔRLU value and the linear equation generated from the standard curve, the cAMP concentration was calculated. Samples were normalized against maximum vs minimum average percentages and subjected to non-linear regression for stimulated Dose response via GraphPad, to assess EC50 in treated human chondrocytes.

### Animal Procedures:

For MMT model, male Lewis rats (n=95 rats+6 extra) were obtained from Envigo RMS, Inc. (Indianapolis, IN), with a mean weight of 262 g. The animals were identified by a distinct mark at the base of the tail delineating group and animal number. After randomization, all cages were labeled with protocol number, group numbers, and animal numbers with appropriate color-coding. During the acclimation and study periods, animals were housed in a laboratory environment with temperatures ranging 19° C. to 25° C. and relative humidity of 30% to 70%. Automatic timers provided 12 hours of light and 12 hours of dark. Animals were allowed access ad libitum to Harlan Teklad Rodent Chow and fresh municipal tap water. Animal care including room, cage, and equipment sanitation conformed to the guidelines cited in the Guide for the Care and Use of Laboratory Animals (Guide, 2011) and the applicable Inotiv Boulder SOPs. Study protocols were approved by Inotiv IACUC standards.

For inflammatory pain assessment, ICR mice were maintained in the SPF unit of the Hebrew University Hadassah Medical School, Jerusalem, Israel. The experimental protocols were approved by the Animal Care Ethical Committee of the Hebrew University-Hadassah Medical School, Jerusalem, Israel. The animals were maintained on a standard pellet diet and water ad libitum. The animals were maintained at a constant temperature (20° C.-21° C.) and a 12 h light/dark cycle.

### Rat Randomization and Husbandry:

Male Lewis rats (n=95 rats+6 extra) were obtained from Envigo RMS, Inc. (Indianapolis, IN), with a mean weight of 262 g. The animals were identified by a distinct mark at the base of the tail delineating group and animal number. After randomization, all cages were labeled with protocol number, group numbers, and animal numbers with appropriate color-coding. During the acclimation and study periods, animals were housed in a laboratory environment with temperatures ranging 19° C. to 25° C. and relative humidity of 30% to 70%. Automatic timers provided 12 hours of light and 12 hours of dark. Animals were allowed access ad libitum to Harlan Teklad Rodent Chow and fresh municipal tap water. Animal care including room, cage, and equipment sanitation conformed to the guidelines cited in the Guide for the Care and Use of Laboratory Animals (Guide, 2011) and the applicable Inotiv Boulder SOPs.

### Rat Model for Medial Meniscal Tear (MMT):

The rat OA model employed was surgically-induced medial meniscal tear (MMT), wherein rats were anesthetized with Isoflurane (VetOne, catalogue No. 502017), and the right knee area was prepared for surgery. A skin incision was made over the medial aspect of the knee, and the medial collateral ligament was exposed by blunt dissection and then transected. The medial meniscus was cut through the full thickness to simulate a complete tear. Skin and subcutis were closed with 4-0 Coated Vicryl (polyglactin 910) Violet Braided Suture (Ethicon, catalogue No. J399H), and slight hand pressure was applied to the wound for approximately 3 minutes for hemostasis. Subcutaneous (SC) dose of buprenorphine (0.05 mg/kg) administered after the animal awakened post-surgery. Rats were weighed daily on study days −1 through 7 and again on days 14, 21, and 28 (prior to necropsy). Dosing was for vehicle and Fenchon 1D (24 μg/mL-high and 8 μg/mL-low concentrations) was initiated at study day 4, and thereafter at days 7, 10, 14, 17, 21, 24. For FGF18 positive control (i.e. expected to exhibit anabolic cartilage response), dosing was initiated at 7 and thereafter at days 14 and 21. The animals were euthanized for necropsy 28 days post-surgery, following Isoflurane anesthesia, and bled to exsanguination followed by bilateral pneumothorax.

### Post Necropathy Histopathology Assessments:

Right knees were collected from all animals and trimmed of muscle and patellae. The trimmed joints were placed in 10% neutral buffered formalin (NBF) for histologic processing and evaluation. Samples were fixed for 3 days in 4% paraformaldehyde and decalcified for 21 days in 10% EDTA, pH=7.5. Then following dehydration in a graded series of ethanol washes, joints were embedded in paraffin and sectioned to 7 μm slices, following trimming off 1 mm until the tibiofemoral compartments are fully observed. Sections were obtained from each knee and stained with toluidine blue (0.04% in 0.2 M acetate buffer, Ph=4.0), based on a modified version of the methods used in Schmitz, et al. (2010), (11). Histopathology was carried out using an ocular micrometer. The following structural histological characterization was carried out:

### Medial Tibial Zonal Cartilage Degeneration Score:

Regional differences across the tibial plateau were taken into consideration by dividing each section into three zones: (1) outside, (2) middle, and (3) inside. In the surgical OA model, the outside (zone-1) and middle (zone-2) thirds are most severely affected, and milder changes are present on the inside third (zone 3). When zones were scored individually, scores were assigned based on percent area of the zone affected. General cartilage degeneration including chondrocyte death/loss, proteoglycan (PG) loss, and collagen loss or fibrillation. Zones were scored individually (0-5; 5 representing sever damage; SD3), and a sum of all three zones was calculated (termed “Total Joint scores”).

### Zonal Depth Ratio of Cartilage:

The zone related cartilage depth is calculated from the projected articular surface tot the tidemark. Reduced ratios embody less detected cartilage width vs projected cartilage with area, per zone.

### Osteophyte Score and Measurement:

Osteophyte thickness (tidemark to furthest point extending toward synovium) was measured and scored according to a range from 0-5, as in SD4.

### Medial Tibial Bone Damage and Sclerosis Scores:

Damage to the calcified cartilage layer and subchondral bone was scored using the criteria in SD5. Generally damage was considered as invagination of deep zone cartilage into the subchondral bone layer.

### Synovitis Score:

Synovial inflammation was scored (evaluation focuses on the lateral side since that is the area uncomplicated by the surgery) as indicated in SD6. Descriptions of other changes (typically fibrosis, or acute inflammation/neutrophil infiltration extending into the lateral compartment usually associated with IA treatments) were also provided, if present.

### Inflammatory Pain Assessment in Mice Paw:

To induce inflammation, 40 μL of 1.5% (w/v) zymosan A (Sigma) suspended in 0.9% saline was injected into the sub-planter surface of the right hind paw of the mice. Immediately after zymosan injection, antagonists were injected intraperitoneally (IP) and after 30 minutes, the fenchone derivatives dissolved in 0.1 mL vehicle containing ethanol:Cremophore:saline at a ratio of 1:1:18 were injected. Control mice were injected with the vehicle only. After 2, 6, and 24 hrs, paw swelling, and pain perception were measured.

Specifically, measurement of Oedema Formation was assessed by monitoring paw swelling via calibrated calipers (0.01 mm), 6 and 24 hrs following injections of zymosan alone and/or the test compounds. Pain hyperalgesia was evaluated by the paw withdrawal von Frey test at 6, and 24 h following injections of zymosan and/or the test compounds. In the von Frey nociceptive filament assay, von Frey calibrated monofilament hairs of logarithmically incremental stiffness (0.008-300 g corresponding to 1.65-6.65 log of force). In this study, only 1.4-60 g corresponding to 4.17 to 5.88 log of force was used, to test the mouse sensitivity to a mechanical stimulus on the swollen paw. The measurements were performed in a quiet room. Before paw pain measurements, the animals were held for 10 sec. The trained investigator applied the filament to the central area of the hind paw with gradual increasing size. The test consisted of poking the middle of the hind paw to provoke a flexion reflex followed by a clear flinch response after paw withdrawal. Each one of the von Frey filaments was applied for approximately 3-4 s to induce the end-point reflex. The first testing was done by using the force filament of 1.4 g. If there was no withdrawal response, the next higher stimulus was tried. The mechanical threshold force (in grams (g)) was defined as the lowest force imposed by two von Frey monofilaments of various sizes, required to produce a paw retraction. The untreated left hind paw served as a control.

### Measurement of Serum TNFα Levels:

Blood was collected 24 h after zymosan injection, and the sera were assayed for TNFα using a mouse TNFα ELISA kit (R&D Systems, Minneapolis, MN, USA), according to the manufacturer's instructions.

### Statistical Analysis:

Group means and standard deviations (SD) were determined for each group. Treatment groups were compared to the vehicle disease control group (Vehicle-MMT) using a Kruskal-Wallis (KW) test with a Dunn's post-hoc analysis for scored (non-parametric) parameters. Sham control rats were compared to the disease control group using a Student's Mann-Whitney U test (non-parametric). Statistical tests were performed using Prism version 9.3.0 software (GraphPad). Statistical significance according to Mann-Whitney or KW is denoted with an asterisk (*) for p<0.05, 2 asterisks (**) for p<0.01, 3 asterisks (***) for p<0.001 and 4 asterisks (****) for p<0.0001.

### Results

### Analysis of Fenchones 1D and 1B Potency and Antagonistic Activity:

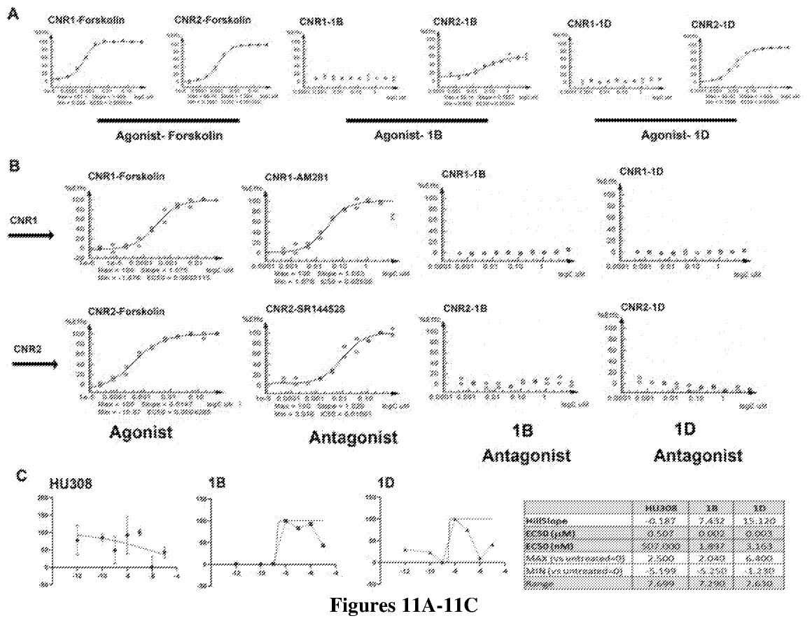

As a first step, the capacity of 1D and 1B compounds to stimulate an agnostic affect by dose dependent exposure of the compounds to cells ether expressing CNR1 or CNR2, was assessed as compared to Forsulin positive control know to stimulate both receptors (FIG. 11A). The results exhibit no response for CNR1 vs a noticeable response for CNR2 (SD1, FIG. 11A). Notably, EC50 for 1B was 0.05 μM vs 0.01 μM for 1D. Next, it was attempted to determine if this is an antagonistic effect of the 1D, 1B molecules which may be a result of Gi activation, for each receptor. To this end, Forskulin was utilized as a positive control while AM281 and SR144528 were utilized as positive controls for CNR1 and CNR2, respectively (FIG. 11B). The data show no detectable antagonistic effect of 1D or 1B for ether receptors, indicating that the 1D/1B compounds are specifically agonistic to CNR2 and exert a Gαs effect increasing cAMP levels within the cells. This was further conformed in chondrocytes isolated from OA patents showing a low EC50 for 1B/1D vs HU308, with a similar potency for both Fenchone compounds (FIG. 11C).

### Assessment of Inflammatory Pain Related Effects of 1D/1B Fenchones:

It was next attempted to utilize a mouse model for inflammatory pain which is induced in mice paws via zymosan SC administration to the paw. Thereafter the compounds were administered IP and mice were monitored for pain and swelling as shown in the experimental setup presented in FIG. 2A. As a first step, it was assessed that the responsiveness of 1D and 1B individually after zymosan induction (FIG. 2B, Left graph), which both exhibited a reduced swelling response after 6h vs control. Similarly, the CB2 antagonist SR144528 had a similar effect in reducing swelling at 6h, when administered individually. However, when administering SR144528 CB2-antagonist with 1B, paw swelling was unaffected. This confirms that the effect of 1B is solely via CB2 agonism. On the other hand, when administering SR144528 CB2-antagonist with 1D, the swelling was reduced compared to the control, indicating that 1D may not solely agonize CB2 and may have other potential anti-inflammatory effects. Interestingly, these effects were not observed 24h after induction, and were also insignificant in von Frey assays (FIG. 2B, Right graph), albeit an increase in gram force displayed in 1D/1B treatment.

Since SR144528 alone reduced swelling, another CB2 antagonist, AM630, which has lesser anti-inflammatory effects compared to SR144528 was next utilized to test 1D in an inflammatory pain model. The data in FIG. 2C (Left graph) display reduced swelling at 6h after administering 1D and AM630, indicating a potential dual affect. Interestingly reduced swelling with 1D alone was sustained even after 24 h and the CB2 antagonist AM630 fully inhibited its effect. These data were in line with the von Frey pain phenotypes showing a significant affect for 1D alone, which was reversed upon addition of AM630 at 24h (FIG. 2C, Right graph). Moreover, the levels of TNF-α were reduced significantly for 1D (25 mg/kg) (FIG. 2D), an effect that was abolished by adding the CB2 antagonist AM630.

### In-Vivo Rat MMT Model:

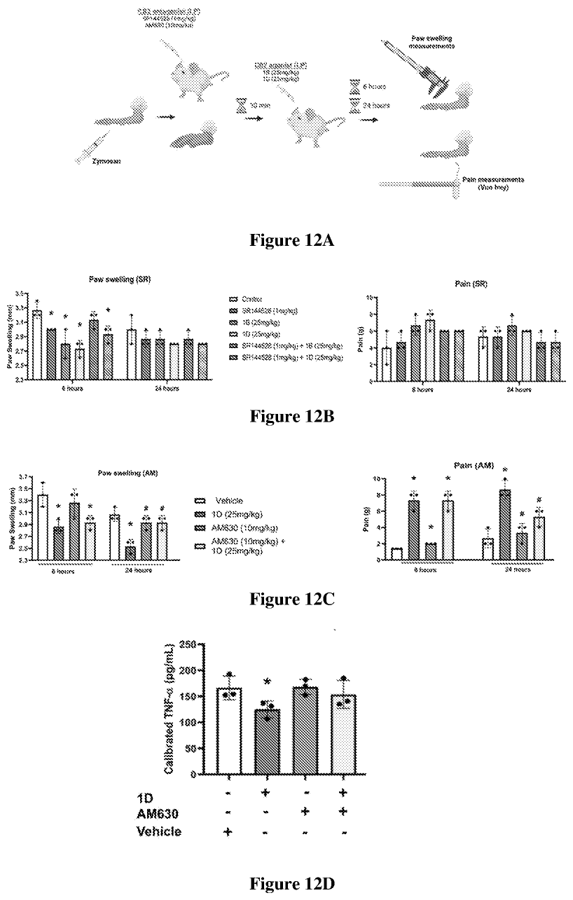

The rat MMT post-traumatic model was employed for right limbs of Lewis rats, with all animals surviving study termination. Sham groups served as a control, while vehicle groups were intra-articularly (IA) injected with the carrier at 4, 7, 10, 14, 17-, 21 and 24-days post MMT, similar to the high and low dose of 1D, which exhibited slightly better EC50 levels (FIG. 12A). As positive control, a FGF18 IA route at 7, 14 and 21 days post MMT was utilized. FGF18 was shown to bestow anabolic effect to articular cartilage and has recently passed phase 2 for DMOAD.

Change in rat weight from 4 days to 28 days post MMT, appeared to be similar with a slight but significant increase for rats with 1D high dose, vs Vehicle and FGF18 groups (FIG. 12B). During the 28 day term, all treatments displayed higher gram force in the contralateral left joint vs sham, which appeared to be most significantly increased in the FGF18 treated rats (FIG. 12C). The cumulative area under the curves for dynamic weight bearing (DWB) exhibited the highest values in the FGF18 treated rats (FIG. 12D), compared to al groups. While low dose 1D showed increased left to right lad bearing vs sham, this was reduced to a similar extent in the high dose 1D which appeared to have statistically insignificant levels vs sham and vehicle treated post MMT. These data highlight a possible placebo effect of the vehicle in this model. While the data do not suggest a dramatic analgesic effect of the high doe 1D, they highlight that its administration didn't incur substantial pain effects to the joint, comparable to FGF18.

### Histopathological Profiling of Post-MMT Joints:

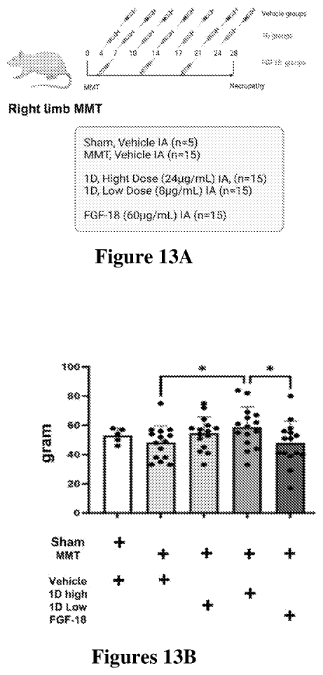

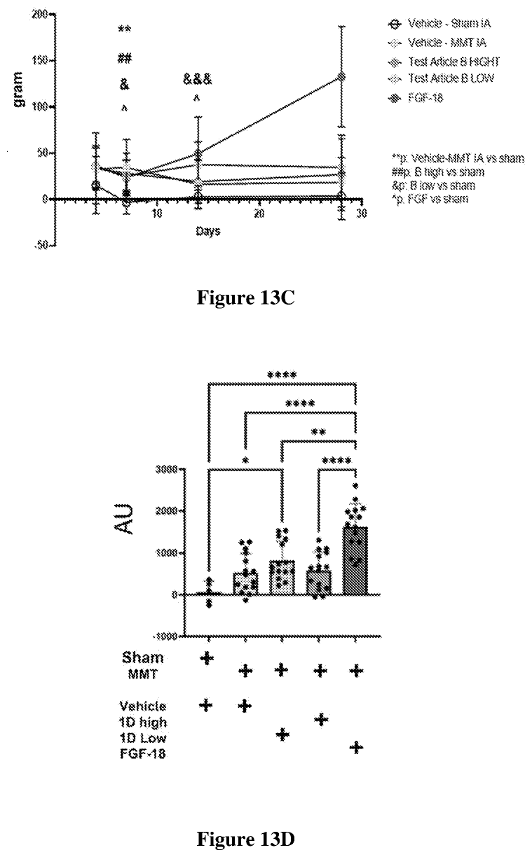

Following 28-days from MMT, joints were assessed for several structural hallmarks of OA. FIG. 13A indicated hallmarks assessed and their graphical illustration. In particular, the zonal and sum of articular cartilage degeneration was assessed for the medial tibial joint compartment (a; SD3). Similarly, Zonal depth ratios were determined based on the ratio of depth detected from the edge to the tidemark, vs the expected depth of the zone (b). For example, significant distraction would exhibit reduced zonal depth ratios. Synovitis scores were assessed according to scoring table (c, SD6). Osteophyte measurements were manually taken and osteophyte scores assessed, based on table (d; SD4). Finally, tibial bone damage was scored based on table (e; SD5). All raw scores are present in an excel sheet under SD8.

### Cartilage Degeneration Scores:

FIGS. 13B and 13C, exhibits that MMT rats treated with vehicle had cartilage damage that was most severe in zone 1, and appeared to be reduced upon FGF18 administration. Similarly, FGF18 exhibited significantly reduced degeneration scores vs all groups, with equivalent scores with the 1D high dose. Interestingly, zones 1 and 2 appear to show a dose effect accompanied with reduced degeneration scores for the high dose of 1D. Finally, zone 3 appeared to show no beneficial effect of FGF18, yet 1D for both high and low dose exhibited less degeneration scores which were statistically significant. The total zonal scores display significantly reduced degenerative scores for 1D high dose and FGF18 compared to the 1D low does and Vehicle groups, post MMT.

### Medial Tibial Depth Ratio:

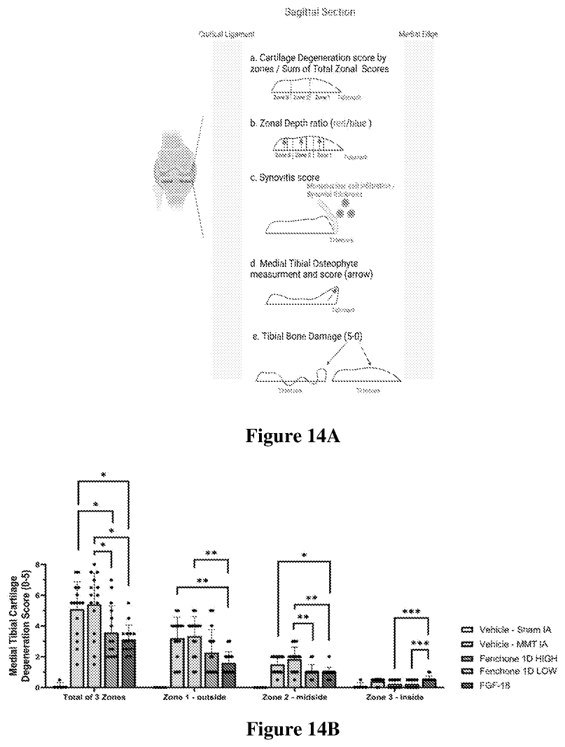

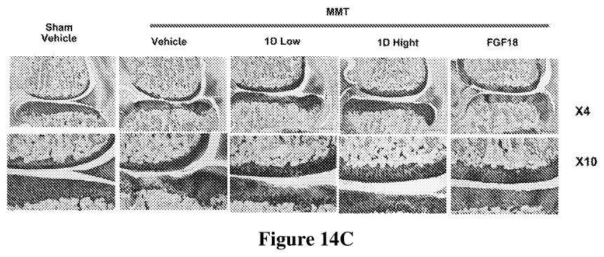

Depth ratios exhibited similar trends as the degenerative scores, displaying significantly lower depth rations for FGF18 treated rats, which were similar to the trend of the 1D high dose, for zone 1 (FIG. 14). Zone 2 exhibited a beneficial dose effect for the high dose of 1D, yet zone 3 displayed higher depth ratios for FGF18 which appeared to be significantly lower in high dose of 1D vs vehicle, and FGF18. The mean zonal depth ratios exhibited reduced depth ratios for FGF18 and high dose 1D, while vehicle and low dose 1D exhibited similar mean depth ratios.

### Assessments of Synovitis and Osteophyte Formation:

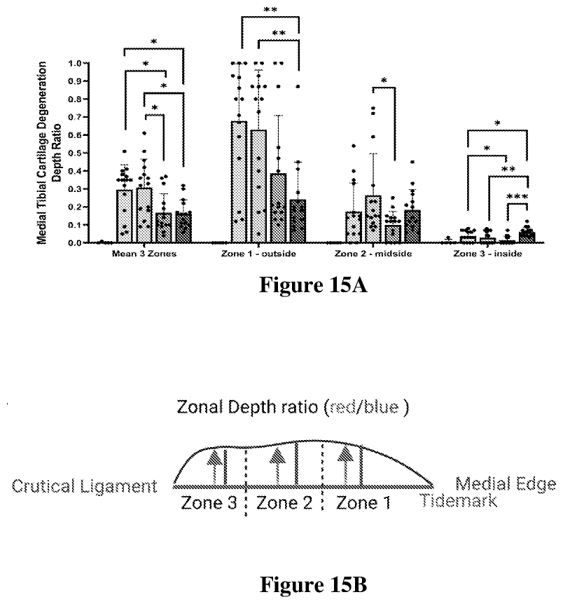

As a next step it was determined that synovitis scores ranging from 0 (i.e. normal synovial histopathology) to 5 (severe synovitis). The data show that medial synovial scores are highest for FGF18 vs all groups (FIG. 15A, arrows on representative images), possibly explain the dynamic load bearing profiles in these rats (FIG. 12C, 12D). Interestingly synovitis scores were higher in the low dose 1D, however the high dose 1D appeared similar to the sham and vehicle synovial scores. The data indicate that high dose of 1D did not incur synovial inflammation.

Osteophyte measurements exhibited significantly higher measurements and scores for FGF18 vs all groups (FIG. 15B, arrows on representative images). While all groups exhibited higher scores and measurements for osteophytes compared to the sham group, the vehicle, 1D high and 1D low doses didn't display any differences.

### Bone Damage:

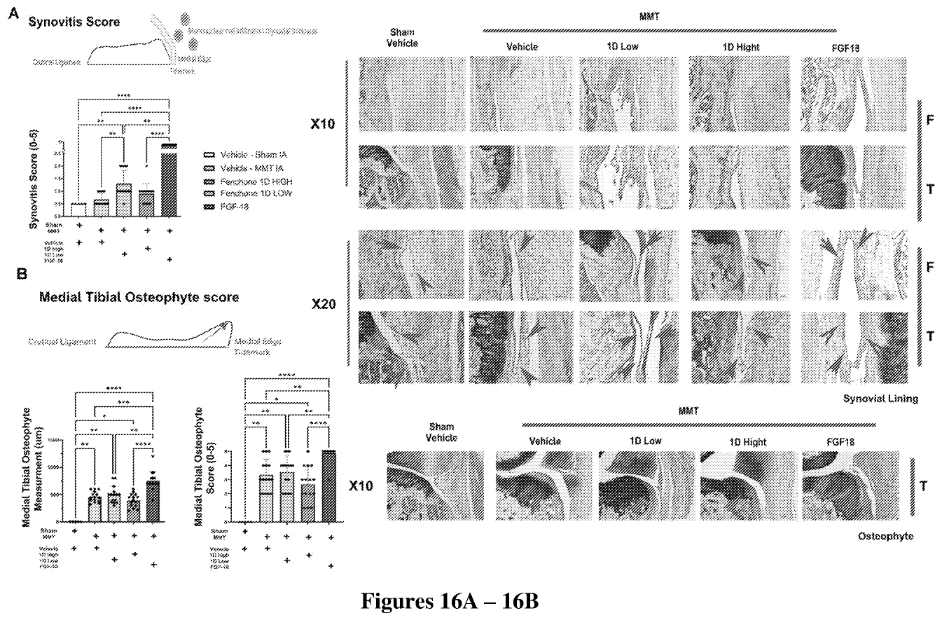

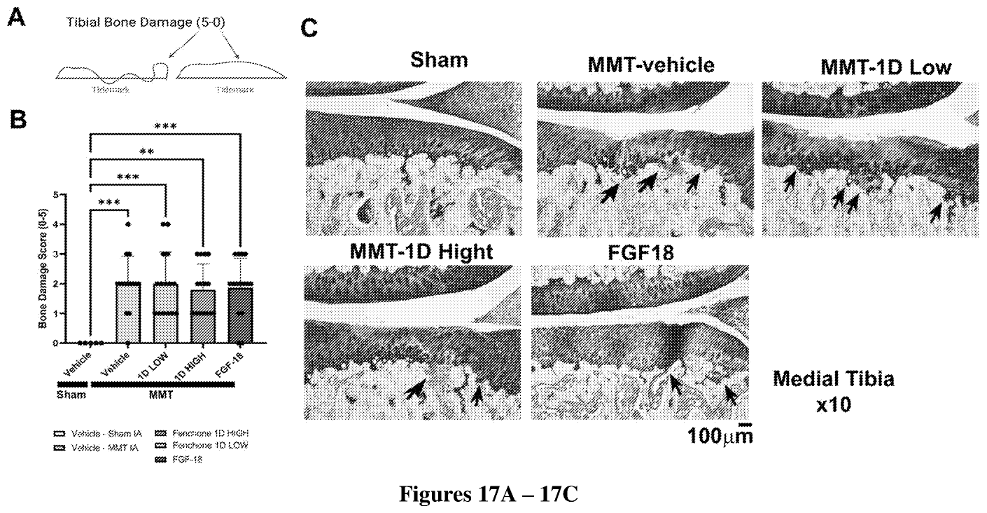

Bone damage is assessed according to invaginated deep zone cartilage into the subchondral area (FIG. 16A illustration), which was increased in all groups vs sham (FIG. 16B), with relatively reduced levels in the FGF18 group vs the 1D high dose (FIG. 16B, 16C). This parameter was not improved amongst all post-MMT subjects.

Cumulatively, the data support that high dose 1D exhibited superior cartilage anabolism which appeared to be similar to that of FGF18 positive control. Moreover, osteophyte and synovitis scores were lower than FGF18, potentially prohibiting the weight bearing shifts observed in dynamic weight bearing. Overall the data appear to show a dose dependent effect of IA administer 1D, which has equivalent DMOAD like effects with no deleterious structural effects to the synovium, or underlying subchondral bone.

### Discussion

The data highlight the potential beneficial effect of a new class of CB2 agonists on cartilage health and potential prevention of OA. While intra-articular administration did not provide an analgesic effect noticeable in dynamic load bearing, it was less detrimental than Fgf18. Moreover, both FGF18 and 1D Fenchone exhibited striking improvement in the preservation of articular cartilage, as judged by the “Join degeneration scores” and “Depth ratio”. THIS structural effect was observed for 1D in a dose dependent manner. Finally, the synovial inflammation, and osteophyte formation potentially affecting the dynamic load bearing in FGF18 treated mice, was not observed in the high dose of 1D, indicating that it may not render any structural alteration that may affect load bearing and pain behaviors. CB2 ablation appeared to cause chondrocyte hypertrophy and may thus potentially contribute to osteophyte formation in OA. While it was not observe that osteophyte in CNR2 null induced to develop OA with age, local IA treatment with HU308 in a DMM, post-traumatic model did reduce osteophyte scores, which is in line with these data.

The local IA use of such CB2 agonist for treatment of OA, was just recently exposed and shows great promise, and is justified due to such mode avoids adverse systemic effects and required 200 fold lesser doses. The effect of 1D/1B exhibits superior EC50 over HU308 in chondrocytes, further accentuating hat lower doses may be efficacious. Particularly to the joint, the current formulation may be further efficacious due to the viscous nature of the intra-articular synovial fluid, which may cause retention of the compound in the joint, to potentiate its biological action. In the mouse, for example, HU308 administration was found to reduce apoptosis, enhance SOX9 levels and PCNA, indicating a strong anabolic effect as a result of tis stimulation. Notably, both HU308 and Fenchonce induce intracellular G-Protein activation rather than repression in chondrocytes, which results in enhanced intracellular cAMP levels. It was shown that the rose of cAMP may contribute to several CREB responsive genes, one of which is SOX9. In previous work CREB activation in osteoblasts, by HU308 was shown to increase cyclin d1 and osteoblast proliferation. Hence the local effect of such CB2 agonists, may be powerful treatments in preventing OA structural decline and maintenance of joint function.

While pain related benefits were not observed in the Dynamic weight bearing model, MMT rarely displays spontaneous alteration in weight bearing, leaving the pain related effects to be explored in other more severe pain models. In particular, MIA models appear to show a significant improvement in pain behaviors when administered with CB2 agonists, as the CB2 agonist JWH133 was reported to improve joint pain thresholds and dynamic weight bearing when applied systemically following MIA in mouse. Similarly, A-796260, a specific CB2 agonist exhibited improved rat hindlimb grip force and when applied systemically post MIA. In fact, CB2 agonist HU308 has been shown to prevent synovial inflammation, which may activate synovial nociceptors in a neuro-immune axis. Therefore, in models of mechanical joint trauma, the neuro-inflammatory axis may not be fully developed to result in baseline pain behaviors, compared to other models as the collagen induced, or MIA models. Of note, HU308 as well as Fenchones 1D and 1B have shown significant improvement in preventing inflammatory pain in a zymozan model, which may be recapitulated in future pain models. In summary use of CB2 agonist compounds, prevented joint damage, inflammation and structural decline, which may effectively prevent OA development for future therapeutic use.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A compound having the general formula (I), including any stereoisomer, salt or solvate thereof: 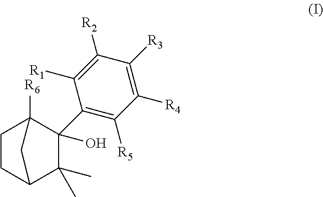
Wherein
R1 and R5 are each selected from H, OH, and —O(straight or branched C1-C5 alkyl);
provided that at least one of R1 and R5 is different than H; and
R2, R3 and R4 are each selected from H, straight or branched C1-C10 alkyl, straight or branched C2-C10 alkenyl, straight or branched C2-C10 alkynyl, halogen; provided that at least one of R2, R3 and R4 are different than H;
R6 is selected from straight or branched C1-C5 alkyl, —CH2OH, —COOH, —COO(straight or branched C1-C5 alkyl), —C(═O)H, —C(═O)(straight or branched C1-C5 alkyl).

2. A compound according to claim 1, wherein R1 and R5 are each O(straight or branched C1-C5 alkyl).

3. A compound according to claim 1, wherein at least one of R1 and R5 is O(straight or branched C1-C5 alkyl).

4. A compound according to claim 1, wherein at least one of R1 and R5 is OH.

5. A compound according to claim 1, wherein at least one of R2, R3 and R4 is halogen.

6. A compound according to claim 1, wherein at least one of R2, R3 and R4 is a straight or branched C3-C10 alkyl.

7. A compound according to claim 1, wherein R6 is a straight or branched C1-C5 alkyl.

8. A compound according to claim 1, wherein R6 is CH3.

9. A compound according to claim 1, wherein R6 is selected from —CH2OH, —COOH, —COO(straight or branched C1-C5 alkyl), —C(═O)H, —C(═O)(straight or branched C1-C5 alkyl.

10. A compound according to claim 1, wherein R1 and R5 are each O(straight or branched C1-C5 alkyl) and R3 is straight or branched C3-C10 alkyl.

11. A compound according to claim 1, wherein R1 and R5 are each O(straight or branched C1-C5 alkyl) and R4 is straight or branched C3-C10 alkyl.

12. A compound according to claim 1, wherein R1 and R5 are each O(straight or branched C1-C5 alkyl) and R2 is a halogen.

13. A compound according to claim 1, wherein R1 and R5 are each O(straight or branched C1-C5 alkyl) and R2 is a halogen and R3 is a straight or branched C3-C10 alkyl.

14. A compound according to claim 1, wherein R1 is O(straight or branched C1-C5 alkyl) and R5 is H.

15. A compound according to claim 1, wherein R1 is O(straight or branched C1-C5 alkyl) and R5 is H and R4 is a straight or branched C3-C10 alkyl.

16. A compound according to claim 1, wherein R1 is O(straight or branched C1-C5 alkyl) and R5 is H and R4 is a straight or branched C3-C8 alkyl.

17. A compound according to claim 1, wherein R1 is O(straight or branched C1-C5 alkyl) and R5 is OH and R3 is a straight or branched C3-C8 alkyl.

18. A compound according to claim 1, selected from:

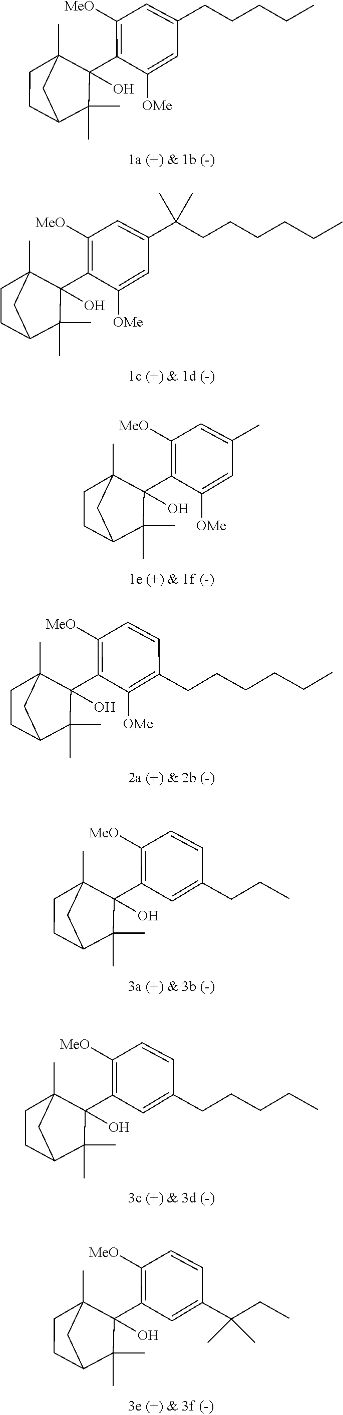

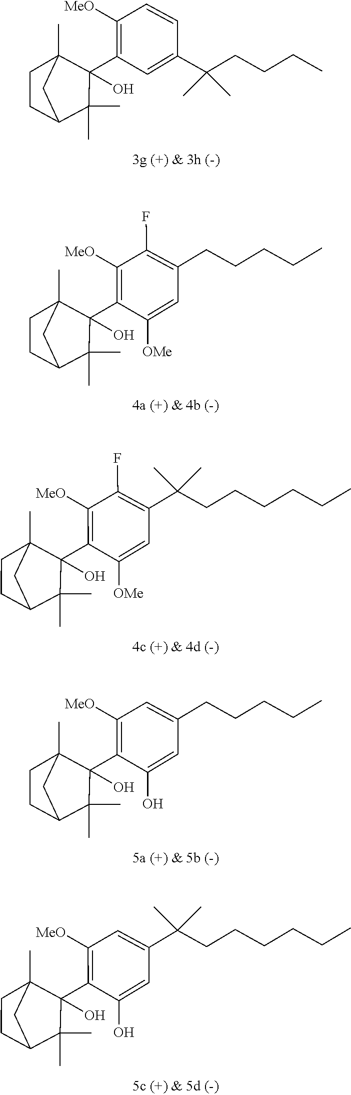 [SMILES: CCCCC(C)(C)c1ccc(OC)c(C2(O)C3(C)CCC(C3)C2(C)C)c1 ; CCCCCc1cc(OC)c(C2(O)C3(C)CCC(C3)C2(C)C)c(OC)c1F ; CCCCCCC(C)(C)c1cc(OC)c(C2(O)C3(C)CCC(C3)C2(C)C)c(OC)c1F ; CCCCCc1cc(O)c(C2(O)C3(C)CCC(C3)C2(C)C)c(OC)c1 ; CCCCCCC(C)(C)c1cc(O)c(C2(O)C3(C)CCC(C3)C2(C)C)c(OC)c1]

19. A pharmaceutical composition comprising at least one compound according to claim 1.

20. (canceled)

21. (canceled)

22. (canceled)

23. A method of treating a subject suffering from a disease, condition or disorder associated with CB2 receptor; said method comprising administering to said subject at least one compound of claim 1.
